(19) United States
(12) Patent Application Publication
(10) Pub. No.: US 2006/0275262 A1
(43) Pub. Date: Dec. 7, 2006

(54) Conditionally replicating viruses and methods for cancer virotherapy

(76) Inventors: James Michael Mathis, Shreveport, LA (US); Arrigo DeBenedetti, Shreveport, LA (US); David T. Curiel, Birmingham, AL (US)

Correspondence Address: FROST BROWN TODD, LLC, 2200 PNC CENTER, 201 E. FIFTH STREET, CINCINNATI, OH, 45202, US

(21) Appl. No.: 11/261,525

(22) Filed: Oct. 28, 2005

Related U.S. Application Data
(63) Continuation-in-part of application No. 10/718,163, filed on Nov. 20, 2003.
Application No. 10/718,163 is a division of application No. 09/916,017, filed on Jul. 26, 2001, now Pat. No. 6,759,394.

(51) Int. Cl. A61K 48/00; C12N 15/861

(52) U.S. Cl. 424/93.2; 514/44; 435/456

(57) ABSTRACT

The present invention provides for methods and compositions for translation of a viral vector both in vitro and in vivo. Specifically, the present invention pertains to a translational control element placed in a vector to cause a selective translation of a viral vector. In one embodiment, the present invention provides for methods and compositions for conditionally expressing a viral vector inside tumor cells, while leaving normal cells unaffected due to their inability to translate the vector

### CROSS-REFERENCE TO RELATED APPLICATION

This application is a continuation-in-part of U.S. patent application Ser. No. 10/718,163, filed Jun. 10, 2004, which is a division of U.S. patent application Ser. No. 09/916,017, filed Jul. 26, 2001, now U.S. Pat. No. 6,759,394, issued Jul. 6, 2004, which applications are hereby incorporated by reference in their entirety.

This invention was made in part with Government support under Grant No. CA69148 awarded by the National Institute of Health. The Government may have certain rights in this invention.

### FIELD OF THE INVENTION

The present invention pertains to a translational control element placed in a vector to cause a selective replication translation of a viral vector. Specifically, the present invention provides for methods and compositions for conditionally expressing a viral gene necessary for vector replication inside tumor cells, while leaving normal cells unaffected due to their inability to replicate the vector.

### BACKGROUND OF THE INVENTION

The major determinant of morbidity and mortality for patients with a primary malignant tumor is the emergence and progression of metastatic islets resistant to conventional therapy. It has been estimated that at least 50% of patients presenting with a primary tumor already bear metastases at the time of diagnosis.

See R. H. Goldfarb et al., “Therapeutic agents for treatment of established metastases and inhibitors of metastatic spread: preclinical and clinical progress,” Current Opinion in Oncology, vol. 4, pp. 1130-41 (1992). Cancer gene therapy has developed as a means of attacking cancers resistant to conventional approaches. Much of the work has been directed at targeting characteristics of the primary tumor, with attention to choice of vector and transcriptional regulation. Two examples of this are the use of tissue-specific promoters and inducible promoters. See K. Binley et al., “An adenoviral vector regulated by hypoxia for the treatment of ischaemic disease and cancer,” Gene Therapy, vol. 6, pp. 1721-1727 (1999). Despite some advances, these approaches have not successfully addressed the major problem of how to target metastases. Not only are metastases more difficult to reach but, due to their heterogeneity, they frequently do not maintain the specific gene expression pattern of the primary tumor, upon which gene therapy is generally designed. See S. J. Hall et al., “Cooperative therapeutic effects of androgen ablation and adenovirus-mediated herpes simplex virus thymidine kinase gene and ganciclovir therapy in experimental prostate cancer,” Cancer Gene Therapy, vol. 6, pp. 54-63 (1999). There is an unfilled need for an effective in vivo cancer gene therapy that permits selective killing of both the primary tumor and distant metastases, while distinguishing cancer cells from normal cells.

One of the main obstacles to gene therapy has been the difficulty of successfully targeting cancer cells, while not harming normal cells. Indeed, it has been found that even when therapeutic vectors are delivered locally to a primary tumor, systemic effects still often occur, indicating that the vector has become blood-borne. See Z. Long et al., “Molecular evaluation of biopsy and autopsy specimens from patients receiving in vivo retroviral gene therapy, Human Gene Therapy, vol. 10, pp.:733-40 (1999); and M. Kaloss et al., “Distribution of retroviral vectors and vector producer cells using two routes of administration in rats,” Gene Therapy, vol. 6, pp. 1389-1396 (1999). One approach to circumvent this problem is to use elements allowing specific transcriptional regulation of the vector, e.g., the use of tissue-specific promoters and inducible promoters. See Binley et al., 1999; and L. M. Anderson et al., “Adenovirus-mediated tissue-targeted expression of the HSVtk gene for the treatment of breast cancer,” Gene Therapy, vol. 6, pp. 854-864 (1999). While these approaches are very promising, they require specific knowledge of the cancer cells, and are not applicable to most situations.

Studies of the molecular mechanisms underlying neoplastic transformation and progression have resulted in the understanding that cancer is a genetic disease, deriving from the accumulation of a series of acquired genetic lesions. Despite advances in chemotherapy, radiation delivery and surgical treatment regimens, survival from many advanced cancers remain poor, and it is apparent that alternative treatment approaches are necessary. Therefore, gene therapy/virotherapy remains a promising strategy for the treatment of cancer. The existing approaches to gene therapy/virotherapy of cancer can be divided into five broad categories: 1) mutation compensation, 2) molecular chemotherapy, 3) genetic immunopotentiation, 4) genetic modulation of resistance/sensitivity and 5) oncolytic therapy or virotherapy. Any of the above gene therapy/virotherapy approaches is fundamentally based on the ability of vector to deliver the therapeutic gene or replication-competent viral genome to target cells with a requisite level of efficiency. A number of characteristics of the Adenovirus type 5 (AdS), including capacity for highly efficient in vivo gene delivery, make it an optimal gene therapy/virotherapy vector suitable for a large number of gene therapy/virotherapy approaches and set Ad5 apart from other vector choices.

### BRIEF SUMMARY OF THE INVENTION

The present invention provides for a conditionally replicating recombinant virus vector, comprising a replicating recombinant virus vector genome, comprising: (a) a replicating recombinant virus vector genome nucleic acid transcription sequence; and (b) a promoter operatively linked to the transcription sequence; wherein the transcription sequence, when transcribed, produces a messenger RNA sequence that comprises an open reading frame encoding a viral protein necessary for replication, and a 5′-untranslated region (5′-UTR) sequence; wherein the untranslated sequence inhibits translation of the viral protein sequence under conditions that exist within normal mammalian cells that do not overexpress eukaryotic initiation factor eIF4E; and wherein the untranslated sequence allows translation of the viral protein sequence under conditions that exist within mammalian cells that overexpress eukaryotic initiation factor eIF4E relative to normal cells.

In one embodiment, the untranslated sequence further comprises a hairpin secondary structure conformation having a stability measured as folded state free energy of ΔG≦about −50 Kcal/Mol.

In another embodiment, the virus vector genome operatively associated with a control sequence. Control sequences operably linked to sequences, i.e. the open reading frame, encoding the protein or peptide of interest, include promoters/enhancers and other expression regulation signals. These control sequences may be selected to be compatible with the host cell for which the expression vector is designed. The term promoter is well-known in the art and encompasses nucleic acid regions ranging in size and complexity from minimal promoters to promoters including upstream elements and enhancers. The promoter is typically selected from promoters that are functional in mammalian cells, although promoters functional in other eukaryotic cells may be used. The promoter is typically derived from promoter sequences of viral or eukaryotic genes. For example, it may be a promoter derived from the genome of the type of cell in which expression is to occur. With respect to eukaryotic promoters, they may be promoters that function in a ubiquitous manner (such as promoters of a-actin, b-actin, tubulin) or, alternatively, a tissue-specific manner (such as promoters of the genes for pyruvate kinase).

In another embodiment, the virus vector genome comprises an adenovirus. In another embodiment, the adenovirus vector is a type 2 or type 5 virus vector. In one embodiment, the E1 coding region is operatively associated with a promoter selected from the group consisting of liver-specific, skeletal muscle-specific, cardiac muscle-specific, smooth muscle-specific, diaphragm muscle-specific, prostate-specific, and/or brain-specific promoters. In one embodiment, the E1 coding region is operatively associated with a cancer cell specific promoter. In another embodiment, the E1 coding region is operatively associated with an inducible promoter. In another embodiment, the recombinant virus vector genome is encapsidated within an virus capsid.

In another embodiment, the present invention provides for a cultured cell comprising the replicating recombinant virus vector of the present invention.

In another embodiment, the present invention provides for a method of introducing a nucleic acid sequence into a cell, comprising contacting a cell with a replicating recombinant virus vector according to the present invention under conditions sufficient for entry of the virus particle into the cell.

In another embodiment, the present invention provides for a method of administering a nucleotide sequence to a subject, comprising administering to a subject a replicating recombinant virus vector according to the present invention in a pharmaceutically acceptable carrier. In another embodiment, the promoter is a cancer cell specific promoter.

The vector may be conditionally regulated by means consisting of a tissue-specific promoter operably linked to an early gene (e.g., E1, E2 and/or E4) and a mutation in an early gene (e.g., E1, E2 and/or E4). Representative tissue-specific promoters are derived from genes encoding proteins such as the prostate specific antigen (PSA), Carcinoembryonic antigen (CEA), secretory leukoprotease inhibitor (SLPI), alpha-fetoprotein (AFP), vascular endothelial growth factor, CXCR4 or survivin.

In another embodiment, the promoter is an inducible promoter. In another embodiment, the promoter is the CXCR4 promoter.

In another embodiment, the present invention provides for a method of treating cancer, comprising administering to a subject that has cancer a composition comprising a replicating recombinant virus vector according to the present invention in a pharmaceutically acceptable carrier; wherein the composition is administered in an therapeutically effective amount and under conditions sufficient for the subject to produce a therapeutic response against the cancer cell.

In another embodiment, the recombinant virus vector is administered by a route selected from the group consisting of oral, rectal, transmucosal, transdermal, inhalation, intravenous, subcutaneous, intradermal, intramuscular, and intraarticular administration. In another embodiment, the recombinant virus vector is injected directly into a cancerous tissue.

In another embodiment, the present invention provides for compositions and methods for using adeno-associated virus for transduction of a target gene in a variety of tissues wherein the expression of the virus is under control of an untranslated sequence; wherein the untranslated sequence inhibits translation of the viral sequence in the absence of eukaryotic initiation factor eIF4E, and wherein the untranslated sequence allows translation of the viral vector sequence into a virus in the presence of eukaryotic initiation factor eIF4E.

In one embodiment, the present invention provides for cancer-specific transcriptional control using a promoter such as that from the human CXCR4 gene that can be used together with cancer-specific translational control utilizing a highly structured 5′-untranslated region (5′-UTR) sequence in the context of a CRAd. In another embodiment, the invention provides for the use of dual-level cancer targeting as means to increase specificity of gene expression in cancers to overcome the problem of non-specific promoter activity (promoter leakage) in normal tissues. In another embodiment, the invention has broad utility to advance cancer-specific CRAds as a novel class of highly specific oncolytic agents to therapeutic clinical trials.

In another embodiment, the present invention provides for dual-targeting of transgene expression to cancer cells using both transcriptional and translational control to substantially enhance Ad5 based cancer virotherapy. This combinatorial approach allows the creation of a highly cancer-specific virotherapy agent, which will be evaluated for the treatment of cancers such as HNSCC.

Conditionally Replicative Adenoviruses (CRAds) represent new promising therapeutic agents applied to cancer treatment. In one embodiment, cancer-specific replication of the CRAds result in viral-mediated oncolysis of infected tumor tissues and release of the virus progeny, capable of further propagating in surrounding tumor cells but not in those of normal tissues, which would be refractory to CRAd replication.

During the past decade utilization of transcriptional control elements (promoters) with cancer-specific induction profiles for expression of adenoviral E1A and E1B genes essential for replication of the virus has led to the development of next generation CRAds that are transcriptionally targeted to cancer cells. Therefore, in one embodiment, an appropriate tumor-specific promoter (TSP) for a given type of cancer is chosen to achieve high cancer specificity of CRAds.

In another embodiment, the present invention shows that the CXCR4 gene promoter not only has a distinct cancer-specific activation profile, but also shows one of the highest activity levels among other TSPs in a panel of selected cancers compared to the ubiquitously utilized Cytomegalovirus (CMV) promoter, particularly in squamous cell carcinomas of the head (HNSCC).

In one embodiment, the background activity of cancer-specific promoters in non-cancer cells is reduced by introducing a second level of cancer targeting for the Ad5E1-genes expression in addition to transcriptional targeting. This can be achieved by placing E1IA gene, which is essential for Ad5 replication function, under cancer-specific translational control via engineering a highly structured 5′-UTR sequence such as that from the Fibroblast Growth Factor 2 (FGF2) upstream of the E1A mRNA coding sequence. In one embodiment, this will render efficient translation of such a chimeric mRNA dependent upon the translation initiation factor eIF4E, which is expressed in limiting amounts in most normal tissues and is over expressed in most cancer cells [1]. It has been previously demonstrated that a FGF25′-UTR - herpes simplex virus thymidine kinase (HSV-Tk) mRNA chimera limits efficient protein translation to tumors but strongly inhibits or even blocks efficient protein translation in normal cells, where eIF4E is the rate-limiting initiation factor [2,3].

In another embodiment, the present invention provides for dual-targeting of transgene expression to cancer cells, namely that this approach augments the cancer targeting strategies currently used alone in AdS based cancer gene therapy/virotherapy. This combinational approach allows the creation of a highly cancer-specific virotherapy agent. In one embodiment, this combinational approach is used for treatment of HNSCC as well as other cancers. Development of such a product also helps to overcome the problem of viral liver toxicity. Thus, in one embodiment, the present invention provides for both the tumor specific promoter and the FGF2 5′-UTR sequence element combined within a single viral agent, such as an adenovirus. Moreover, another embodiment utilizes the CXCR4 promoter and the FGF2 5′-UTR sequence element as a TSP in the context of CRAd.

Squamous cell carcinoma of the head and neck region (HNSCC) is the sixth most frequent cancer worldwide, comprising almost 50% of all malignancies in some developing nations. In the United States, 30,000 new cases and 8,000 deaths are reported each year [63]. Survival rates vary depending on tobacco and alcohol consumption, age, gender, ethnic background, and geographic area. This variability reflects the multifactoral pathogenesis of the disease. Early detection and diagnosis has increased survival but the overall 5-year rate of 50% is among the lowest of the major cancers [64]. Thus, an effective alternative in the treatment of malignant diseases, including but not limited to HNSCC, is greatly needed. On Oct. 16, 2003, China became the first country to approve the commercial production of a gene therapy product, using adenovirus delivery of p53 to treat HNSCC to SiBiono GenTech, a Shenzhen, China based company [65]. Introgen Therapeutics in the U.S. has been using a similar strategy for HNSCC and their Ad-p53 product is showing encouraging results in Phase 3 trials [66]. These outcomes indicate that HNSCC is very amenable to gene therapy treatment approaches. However, the clinical outcome for patients treated with Ad-p53 is unknown and will require long-term follow up.

In one embodiment, the present invention now provides for a combination of transcription and translation control to enhance cancer-specific replication of the CRAds, the major advantage of which is to achieve efficient tumor cell oncolysis and to mitigate tumor cell infection limitations. This ability to target both primary and metastatic lesions means that our approach has wide application in the treatment of many forms of cancer, beyond p53 mutation-positive cancers.

The expression of a virus vector is translationally repressed in normal cells by placing a complex 5′-UTR sequence in front of the vector reading frame.

The invention also provides formulations comprising the recombinant adenoviruses according to the invention that can be used to preserve the recombinant adenoviruses and to administer the recombinant adenoviruses to cells. In one variation, the formulations are used to administer the recombinant adenoviruses to cells in vitro, in another variation the formulations are used to administer the recombinant adenoviruses to cells in vivo.

The invention furthermore provides methods to administer the formulations according to the invention to cells, leading to infection of the cells with the recombinant adenoviruses of the invention. In one variation, the methods are used to administer the formulations to cells in vitro, in another variation the methods are used to administer the formulations to cells in vivo.

The invention also provides compositions of the recombinant adenoviruses according to the invention and cells in which the recombinant adenoviruses according to the invention induce accelerated cell lysis and/or a faster release of virus progeny, compared to recombinant adenoviruses lacking coding sequences for the restoring factor according to the invention. In a preferred variation of the invention, the cells are cancer cells and the cell lysis is oncolysis. In a further preferred variation of the invention, the cells are human cells.

In another embodiment, the invention provides compositions of the recombinant adenoviruses according to the invention and tumors in which the recombinant adenoviruses according to the invention induce accelerated cell lysis and/or a faster release of virus progeny, compared to recombinant adenoviruses lacking coding sequence for the restoring factor according to the invention. In this aspect of the invention, it is preferred that the accelerated cell lysis and/or a faster release of virus progeny results in an accelerated lateral spread by the recombinant adenoviruses from infected cells to neighboring cells in the tumors, compared to recombinant adenoviruses lacking coding sequence for the restoring factor according to the invention. In this aspect of the invention, it is furthermore preferred that the accelerated cell lysis, faster release of virus progeny and/or accelerated lateral spread lead to a more effective destruction or growth inhibition of the tumors. In a preferred variation of the invention, the tumors are growing in an animal body. I In a further variation, the animal body is a human body.

In one embodiment, the modified virus is administered by injection into or near the solid neoplasm. In another embodiment, the modified virus is administered intravenously into the mammal. In another embodiment, the modified virus is administered intraperitoneally into the mammal. In another embodiment, the mammal is immunocompetent. In another embodiment, the modified virus is encapsulated in a micelle or liposome. In another embodiment, the modified virus is administered with an effective amount of an anti-antivirus antibody. In another embodiment, approximately 1 to 1015 plaque forming units of modified virus/kg body weight are administered. In another embodiment, the modified virus is administered in a single dose. In another embodiment, the modified virus is administered in more than one dose. In another embodiment, the administration further comprises the administration of an effective amount of a chemotherapeutic agent.

This invention is directed to a method for treating a cell proliferative disorder in a mammal, comprising administering to proliferating cells in a mammal in an effective amount of one or more viruses selected from the group consisting of modified adenovirus, modified HSV, modified vaccinia virus and modified parapoxvirus orf virus under conditions which result in substantial lysis of the proliferating cells.

The virus may be modified such that the virion is packaged in a liposome or micelle, or the proteins of the outer capsid have been mutated. The virus can be administered in a single dose or in multiple doses. The cell proliferative disorder may be a neoplasm. Both solid and hematopoietic neoplasms can be targeted.

Also provided is a method of treating a neoplasm in a human, comprising administering to the neoplasm an effective amount of virus selected from the group consisting of modified adenovirus, modified HSV, modified vaccinia virus and modified parapoxvirus orf virus, to result in substantial oncolysis of the neoplastic cells. The virus may be administered by injection into or near a solid neoplasm.

Also provided is a method of inhibiting metastasis of a neoplasm in a mammal, comprising administering to the neoplastic cells in a mammal a virus selected from the group consisting of modified adenovirus, modified HSV, modified vaccinia virus and modified parapoxvirus orf virus, in an amount sufficient to result in substantial lysis of the neoplasm.

Also provided is a method of treating a neoplasm in a mammal, comprising surgical removal of the substantially all of the neoplasm and administration of a virus selected from the group consisting of modified adenovirus, modified HSV, modified vaccinia virus and modified parapoxvirus orf virus, to the surgical site in an amount sufficient to result in substantial oncolysis of any remaining neoplasm.

Also provided is a pharmaceutical composition comprising a virus selected from the group consisting of modified adenovirus, modified HSV, modified vaccinia virus and modified parapoxvirus orf virus, a chemotherapeutic agent and a pharmaceutically acceptable excipient.

Also provided is a pharmaceutical composition comprising a virus selected from the group consisting of modified adenovirus, modified HSV, modified vaccinia virus and modified parapoxvirus orf virus, and a pharmaceutically acceptable excipient.

Further, this invention includes a kit comprising a pharmaceutical composition comprising a virus selected from the group consisting of modified adenovirus, modified HSV, modified vaccinia virus and modified parapoxvirus orf virus, and a chemotherapeutic agent.

Additionally, this invention provides a kit comprising a pharmaceutical composition comprising a virus selected from the group consisting of modified adenovirus, modified HSV, modified vaccinia virus and modified parapoxvirus orf virus and an anti-antivirus antibody.

Also provided is a method for treating a population of cells comprising a neoplasm in vitro comprising administering to the population of cells in vitro a virus selected from the group consisting of modified adenovirus, modified HSV, modified vaccinia virus and modified parapoxvirus orf virus in an amount sufficient to result in substantial lysis of the neoplasm.

The invention is also directed to methods of treating a proliferative disorder in a mammal, by immunosuppressing, immunoinhibiting or otherwise rendering the mammal immunodeficient and, concurrently or subsequently, administering a virus selected from the group consisting of modified adenovirus, modified HSV, modified vaccinia virus and modified parapoxvirus orf virus in an amount sufficient to result in substantial lysis of the neoplasm.

In the methods of the invention, modified virus is administered to proliferating cells in mammal. Representative types of modified virus include adenovirus, HSV, parapoxvirus orf virus, or vaccinia virus, which infect humans. In a preferred embodiment, modified adenovirus is used.

The virus may be a recombinant virus from two or more types of viruses with differing pathogenic phenotypes such that it contains different antigenic determinants thereby reducing or preventing an immune response by a mammal previously exposed to a virus subtype. Such recombinant virions can be generated by co-infection of mammalian cells with different subtypes of virus with the resulting resorting and incorporation of different subtype coat proteins into the resulting virion capsids.

The invention furthermore provides methods to construct the recombinant viruses according to the invention and to produce the formulations and compositions according to the invention.

The invention furthermore contemplates the use of the recombinant viruses, methods and formulations according to the invention for the treatment of a disease which involves inappropriate cell survival, where it is preferred that the disease is a disease in a human being. In a particular embodiment of the invention the disease is cancer.

Hereinafter, in several embodiments of the invention a number of ways to provide the recombinant viruses, formulations, methods, compositions, and uses are given. It is to be clearly understood that the description is not meant to in any way limit the scope of the invention as was explained in general terms above. Skilled artisans will be able to transfer the teachings of the present invention to other recombinant viruses, restoring factors, formulations, methods, compositions, and uses that are not mentioned specifically herein without departing from the present invention.

It is also to be understood that the invention includes all combined uses of the recombinant viruses, formulations, methods and compositions of the invention together with other methods and means to kill a population of cells, including but not limited to irradiation, introduction of genes encoding toxic proteins, such as for example toxins or prodrug converting enzymes, and administration of chemical compounds, antibodies, receptor antagonists, and the like.

The definitions of the terms used in the invention specification and claims are deemed either to be sufficiently defined herein or otherwise being clearly understood in the art. Further, any nucleic acid or amino acid sequence of factors/proteins described herein are known sequences, wherein reference is made to commonly available sequence databanks, such as the databanks of EMBL, Heidelberg, Germany, and GenBank (NCBI), both herein incorporated by reference.

Hereinafter, the invention will be further exemplified by the following examples and figures . The examples show a number of ways to provide the recombinant viruses, formulations, methods, compositions, and uses according to the invention. It is to be clearly understood that the examples are not meant to in any way limit the scope of the invention as was explained in general terms above. Skilled artisans will be able to transfer the teachings of the present invention to other recombinant viruses, functional proteins, formulations, methods, compositions, and uses without departing from the present invention.

These and other embodiments of the subject invention will readily occur to those of ordinary skill in the art in view of the disclosure herein.

### BREIF DESCRIPTION OF THE FIGURES

FIG. 1 is a graphical depiction of conditionally replicative virus based therapy. In conventional non-replicative vector based-gene therapy, the vector enters the target cell and expresses the effector gene to kill the tumor cells. In replicative virus-based therapy, after entry, the virus replicates primarily in the infected target cell and kills the cell by cytolysis as a consequence of lytic infection. Then, the released virus infects surrounding target cells. The achievement of this lateral spread is a key event for the effectiveness of replicative virus-based therapy.

FIG. 2 depicts a graphical model of translation initiation. The initiation process is comprised of three steps: 1) formation of the 43S complex, composed of a 40S ribosomal subunit and the initiation factors eIF-2, eIF-3, Met-tRNAi and GTP; 2) formation of the 48S complex containing mRNA; and 3) joining of the 60S subunit to form the complete 80S complex.

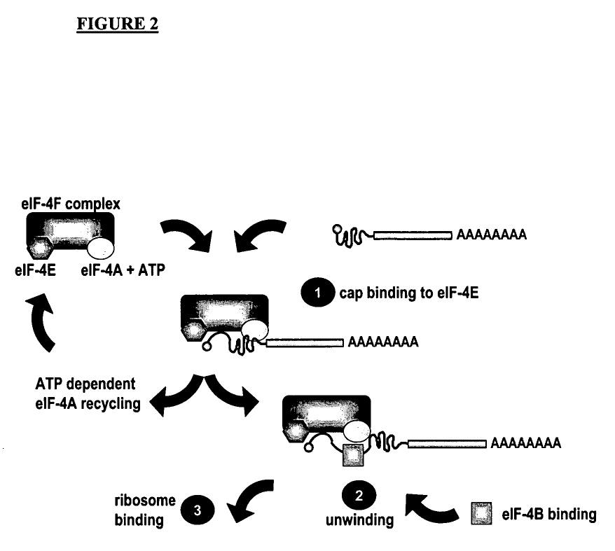

FIG. 3 is a graphical depiction of construction of shuttle vectors are an intermediate step in the production of adenoviruses with single and/or dual-level expression control of the luciferase reporter gene or the E1A gene. Shuttle vectors in which transcription of the E1A or the luciferase genes are controlled by the cancer-specific CXCR4 gene promoter. Shuttle vectors with CXCR4 promoter-controlled transcription of the E1A or the luciferase gene and the FGF2 5′-UTR element in front of the coding sequences. Control shuttle vectors with the CMV promoter-driven transcription of E1A gene or the luciferase gene with or without the FGF25′-UTR element.

FIG. 4 shows endogenous CXCR4 and eIF4E Expression in HNSCC Tumors and Adjacent Surgical Margins. Samples of paired HNSCC tumors and adjacent surgical margin tissues from three patients were assayed for eIF4E and CXCR4 expression by Western blot analysis and compared to expression in the HeLa and FaDu cell lines.

FIG. 5 is a Western blot analysis of E1A protein levels. Western blot analysis of E1A protein expression was performed using lysates from for each cell line used (MCF-10A, and MCF-10A-4E) and infected (at an m.o.i. of 100 p.f.u./cell) with a wild-type Ad vector containing a deletion of the E3 gene (Ad-wt-dE3) or Ad vectors containing the gene for green fluorescent protein (Ad-CMV-GFP), E1A (Ad-CXCR4-E1A), or the 5′-UTR-modified E1A (Ad-CXCR4-UTR-E1A).

FIG. 6. is an in vitro oncolysis assay. Two cell lines were used: a normal breast epithelial cell line expressing low levels of eIF4E (MCF-10A), and the MCF-10A-4E cell line transfected with a G418-selectable plasmid expression vector that constitutively expresses high levels of eIF4E. The cell lines were infected with increasing multiplicities of infection (m.o.i.) from 0.1 to 100 of the indicated adenovirus, and the number of cells adherent after 10 days from infection were visualized by crystal violet staining of the viable cells attached to the wells.

FIG. 7. is an in vitro oncolysis assay. Two cancer cell lines (ZR-75-1 and MDA-MB-231) were infected with increasing multiplicities of infection (M.O.I.) from 0.1 to 100 of the indicated adenovirus, and the number of cells adherent after 10 days from infection were visualized by crystal violet staining of the viable cells attached to the wells.

### DETAILED DESCRIPTION OF THE INVENTION

The practice of the present invention will employ, unless otherwise indicated, conventional methods of virology, microbiology, molecular biology and recombinant DNA techniques within the skill of the art. Such techniques are explained fully in the literature.

The present invention will now be described with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

All publications, patent applications, patents, and other references mentioned herein are incorporated by reference in their entirety. Also incorporated by reference is the full disclosure of the following document and unpublished manuscripts: Robert J. DeFatta, “The Eukaryotic Translation Initiation Factor (eIF) 4E During Cancer Progression and as a Target for Cancer Gene Therapy,” a Dissertation, submitted to the Graduate Faculty of Medical Center of Louisiana State University and Agricultural and Mechanical College, catalogued and placed on the shelf on Mar. 20, 2001; DeFatta, R J, Li, Y., and De Benedetti, A. (2002) Selective killing of cancer cells based on translational control of a suicide gene. Cancer Gene Therapy 9: 573-578. DeFatta, R J., Chervenak, R P., and De Benedetti, A. (2002) A cancer gene therapy approach through translational control of a suicide gene. Cancer Gene Therapy 9: 505-512. In the event of an otherwise irreconcilable conflict, however, the present specification shall control.

Except as otherwise indicated, standard methods may be used for the construction of the recombinant adenovirus genomes, helper adenoviruses, and packaging cells according to the present invention. Such techniques are known to those skilled in the art. See, e.g., SAMBROOK et al., MOLECULAR CLONING: A LABORATORY MANUAL 2nd Ed. (Cold Spring Harbor, N.Y., 1989); F. M. AUSUBEL et al. CURRENT PROTOCOLS IN MOLECULAR BIOLOGY (Green Publishing Associates, Inc. and John Wiley & Sons, Inc., New York).

By “adenovirus vector” is meant a vector derived from an adenovirus serotype, including without limitation, to any of the fifty distinct serotypes of human adenoviruses have been identified to date. These serotypes have been classified into six subgroups (A-F) based on sequence comparisons, each subgroup with different tropisms. Thus, an adenovirus vector is defined herein to include at least those sequences required in cis for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging.

By “adenovirus virion” is meant a complete virus particle, such as a wild-type (wt) adenovirus particle (comprising a linear, single-stranded adenovirus nucleic acid genome associated with a capsid protein coat). In this regard, single-stranded viral nucleic acid molecules of either complementary sense, e.g., “sense” or “antisense” strands, can be packaged into any one virion and both strands are equally infectious.

An “Adenovirus vector genome” or “Ad vector genome” refers to the viral genomic DNA, in either its naturally occurring or modified form. A “rAd vector genome” is a recombinant Ad genome (i.e., VDNA) that comprises one or more heterologous nucleotide sequence(s). The Ad vector genome or rAd vector genome will typically comprise the Ad terminal repeat sequences and packaging signal. An “Ad particle” or “rAd particle” comprises an Ad vector genome or rAd vector genome, respectively, packaged within an Ad capsid. Generally, the Ad vector genome is most stable at sizes of about 28 kb to 38 kb (approximately 75% to 105% of the native genome size). In the case of an adenovirus vector containing large deletions and a relatively small transgene, “stuffer DNA” can be used to maintain the total size of the vector within the desired range by methods known in the art.

“Adenovirus” is a double stranded DNA virus of about 3.6 kilobases. In humans, adenoviruses can replicate and cause disease in the eye and in the respiratory, gastrointestinal and urinary tracts. The term “adenovirus” as used herein is intended to encompass all adenoviruses, including the Mastadenovirus and Aviadenovirus genera. To date, at least forty-seven human serotypes of adenoviruses have been identified (see, e.g., Fields et al., Virology, volume 2, chapter 67 (3d ed., Lippincoft-Raven Publishers). Preferably, the adenovirus is a serogroup C adenovirus, still more preferably the adenovirus is serotype 2 (Ad2) or serotype 5 (Ad5). The various regions of the adenovirus genome have been mapped and are understood by those skilled in the art (see, e.g., FIELDS et al., VIROLOGY, volume 2, chapters 67 and 68 (3d ed., Lippincoft-Raven Publishers). The genomic sequences of the various Ad serotypes, as well as the nucleotide sequence of the particular coding regions of the Ad genome, are known in the art and may be accessed, e.g., from GenBank and NCBI (See, e.g., GenBank Accession Nos. J091, M73260, X73487, AF108105, L19443, NC 003266 and NCBI Accession Nos. NC 001405, NC 001460, NC 002067, NC 00454). Those skilled in the art will appreciate that the inventive adenovirus vectors may be modified or “targeted” as described in Douglas et al., (1996) Nature Biotechnology 14:1574; U.S. Pat. No. 5,922,315 to Roy et al.; U.S. Pat. No. 5,770,442 to Wickham et al.; and/or U.S. Pat. No. 5,712,136 to Wickham et al.

“Administration to a proliferating cell or neoplasm” indicates that the virus is administered in a manner so that it contacts the proliferating cells or cells of the neoplasm (also referred to herein as “neoplastic cells”).

The term “cancer” or “neoplasm” as used herein refers to malignant tumor that metastasize and proliferate immortally. Cancer is a group of diseases classified by the tissues affected, and include, but are not limited to breast cancer, prostate cancer, ovarian cancer, malignant hepatoma, carcinoma of esophagus, lung cancer, cancer of rectum, nasopharyngeal carcinoma, carcinoma of stomach, pleural effusion, carcinoma of ovarium, ascites, and melanoma.

The term “cancer therapy” as used herein refers to that vectors for infecting cancer cells, so as to destroy cancer cells. Such vectors may optionally include one or more therapeutic gene. The therapeutic genes include genes related to cell apoptosis, cell lysis, cell suicide, etc.

As used herein, the terms “conditionally regulated” and “conditionally-replicative” refer to the expression of a viral gene or the replication of a virus or a vector, wherein the expression of replication is dependent (i.e., conditional) upon the presence or absence of specific factors in the target cell.

As used herein, the term “cytokine” refers to all small proteins with the properties of locally acting hormones. They serve to communicate between cells in a paracrine manner, and may also act in an autocrine manner on the same cell that produces the cytokine(s). Growth factors are types of cytokines that are anti-arthritic in that they maintain synthesis of the cartilaginous matrix. Growth factors include, but are not limited to, transforming growth factor (TGF), TGF-β, TGF-β2 and TGF-β3, fibroblast growth factor (FGF), αFGF and βFGF, insulin-like growth factor (IGF) IGF-1 and IGF-2. Growth hormone, and at least some of the bone morphogenetic proteins (BMP) are also cytokines.

The phrase “delivering a gene” or “transferring a gene” refers to methods or systems for reliably inserting foreign DNA into host cells, such as into cells. Such methods can result in transient or long-term expression of nonintegrated transferred DNA, extrachromosomal replication and expression of transferred replicons (e.g., episodes), or integration of transferred genetic material into the genomic DNA of recipients. Gene transfer provides a unique approach for the treatment of acquired and inherited diseases. A number of systems have been developed for gene transfer into mammalian cells. See, e.g., U.S. Pat. No. 5,399,346.

By “DNA” is meant a polymeric form of deoxyribonucleotides (adenine, guanine, thymine, or cytosine) in double-stranded or single-stranded form, either relaxed and supercoiled. This term refers only to the primary and secondary structure of the molecule, and does not limit it to any particular tertiary forms. Thus, this term includes single- and double-stranded DNA found, inter alia, in linear DNA molecules (e.g., restriction fragments), viruses, plasmids, and chromosomes. In discussing the structure of particular DNA molecules, sequences may be described herein according to the normal convention of giving only the sequence in the 5′ to 3′ direction along the nontranscribed strand of DNA (i.e., the strand having the sequence homologous to the mRNA). The term captures molecules that include the four bases adenine, guanine, thymine, or cytosine, as well as molecules that include base analogues which are known in the art.

“eIF4E” refers to the cap-binding sub-unit of the eIF4E complex. eIF4E binds the mRNA cap structure and forms eIF4F complexes that recruit 40S subunits to the mRNA. The eukaryotic initiation factor 4E (eIF4E) is a component of the cellular translational apparatus. Translation initiation on eukaryotic mRNA includes the recruitment of the 40S ribosomal subunit to the 5′ end of mRNA. This is mediated by eukaryotic translation initiation complex 4F (eIF4F) that is a heterotrimetic complex containing eIF4E, eIF4A, and eIF4G. eIF4A is an RNA-dependent RNA helicase which unwinds mRNA secondary structure and eIF4G is a large polypeptide containing binding sites for eIF4E, eIF4A, eIF3 and poly(A) binding protein. eIF4E facilitates the initiation of translation by directly binding to the mRNA 5′ cap structure (m⁷ GpppN). The sequence of DNA encoding human eIF4E has been determined [Reychlik, W. et al. (1987) Proc. Natl. Acad. USA 84:945-949]. Yeast eIF4E and a fusion protein of mouse eIF4E have been expressed in E. coli [Edery, I., et al. (1998) Gene 74:517-525; Edery, I., et al. (1995) Mol. Cell. Biol. 15:3363-3371]. Haas, D. W. et al. (1991) Arch. Biochem. Biophys. 284:84-89 reported purification of native eIF4E from erythrocytes. Stem, B. D. et al. (1993) reported isolation of recombinant eIF4E using denaturing concentrations of urea.

“Expression control element”, or simply “control element”, refers to DNA sequences, such as initiation signals, enhancers, promoters and silencers, which induce or control transcription of DNA sequences with which they are operably linked. Control elements of a gene may be located in introns, exons, coding regions, and 3′ flanking sequences. Some control elements are “tissue specific”, i.e., and affect expression of the selected DNA sequence preferentially in specific cells (e.g., cells, of a specific tissue), while others are active in many or most cell types. Gene expression occurs preferentially in a specific cell if expression in this cell type is observably higher than expression in other cell types.

A “gene” or “coding sequence” or a sequence which “encodes” a particular protein, is a nucleic acid molecule which is transcribed (in the case of DNA) and translated (in the case of mRNA) into a polypeptide in vitro or In vivo when placed under the control of appropriate regulatory sequences. The boundaries of the gene are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the gene sequence.

“Herpes simplex virus” (HSV) refers to herpes simplex virus-1 (HSV-1) or herpes simplex virus-2 (HSV-2). HSV gene gammal 34.5 encodes the gene product infected-cell protein 34.5 (ICP34.5) that can prevent the antiviral effects exerted by PKR. ICP34.5 has a unique mechanism of preventing PKR activity by interacting with protein phosphatase 1 and redirecting it activity to dephosphorylate eIF-2alpha.29 In cells infected with either wild-type or the genetically engineered virus from which the gammal 34.5 genes were deleted, eIF-2alpha is phosphorylated and protein synthesis is turned off in cells infected with gammal 34.5 minus virus. It would be expected that the gammal 34.5 minus virus would be replication competent in cells with an activated Ras pathway in which the activity of ICP34.5 would be redundant. HSV is unable to replicate in cells which do not have an activated Ras-pathway. Thus, HSV can replicate in cells which have an activated Ras-pathway.

The term “heterologous” as it relates to nucleic acid sequences such as gene sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention. Allelic variation or naturally occurring mutational events do not give rise to heterologous DNA, as used herein.

A “heterologous nucleotide sequence” or “heterologous nucleic acid sequence” will typically be a sequence that is not naturally-occurring in the virus. Alternatively, a heterologous nucleotide or nucleic acid sequence may refer to a viral sequence that is placed into a non-naturally occurring environment (e.g., by association with a promoter with which it is not naturally associated in the virus).

“Homology” refers to the percent of identity between two polynucleotide or two polypeptide moieties. The correspondence between the sequence from one moiety to another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions that form stable duplexes between homologous regions, followed by digestion with single-stranded-specific nuclease(s), and size determination of the digested fragments. Two DNA, or two polypeptide sequences are “substantially homologous” to each other when at least about 80%, preferably at least about 90%, and most preferably at least about 95% of the nucleotides or amino acids match over a defined length of the molecules, as determined using the methods above.

By “infectious”, as used herein, it is meant that the virus can enter the cell by natural transduction mechanisms and express the transgene therein. Alternatively, an “infectious” virus is one that can enter the cell by other mechanisms and express the transgene therein. As one illustrative example, the vector can enter a target cell by expressing a ligand or binding protein for a cell-surface receptor in the adenovirus capsid or by using an antibody(ies) directed against molecules on the cell-surface followed by internalization of the complex, as is described hereinbelow.

“Initiator” refers to a short, weakly conserved element that encompasses the transcription start site and which is important for directing the synthesis of properly initiated transcripts.

A “mammal suspected of having a proliferative disorder” means that the mammal may have a proliferative disorder or tumor or has been diagnosed with a proliferative disorder or tumor or has been previously diagnosed with a proliferative disorder or tumor, the tumor or substantially all of the tumor has been surgically removed and the mammal is suspected of harboring some residual tumor cells.

By “mammalian subject” is meant any member of the class Mammalia including, without limitation, humans and non-human primates such as chimpanzees and other apes and monkey species; farm animals such as cattle, sheep, pigs, goats and horses; domestic mammals such as dogs and cats; laboratory animals including rodents such as mice, rate and guinea pigs, and the like. The term does not denote a particular age or sex. Thus, adult and newborn subjects, as well as fetuses, whether male or female, are intended to be covered.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring. As used herein, the term “recombinant” indicates that a polynucleotide construct (e.g., and adenovirus genome) has been generated, in part, by intentional modification by man.

The term “oncolytic techniques” as used herein refers to all kinds of effective protocols that can induce the lysis or death of tumor cells including apoptosis and necrosis. These protocols include application of an oncolytic virus that lead to the lysis or death of cancer cells.

The term “oncolytic virus” as used herein refers to a genetically engineered virus that may replicate immortally in cancer cells, so as to kill these cancer cells. Adenovirus dl1520 is an example of oncolytic viruses. The oncolytic viruses referred to in this invention could be herpes simplex virus (HSV-1), adenovirus, newcastle disease virus (“NDV”), poliovirus, measles virus, vesicular stomatitis virus (“VSV”), etc. In one embodiment, the lytic viruses include other viruses, for example, baculovirus; members of the Herpesviridae such as HSV1, HSV2, VZV, HCMV, HHV8; parvoviruses such as B19, AAV-2; members of the Togaviridae including alphaviruses such as equine encephalitis viruses & Sindbis and rubiviruses such as rubella; polyoma viruses such as SV40; arboviruses such as Getah arbovirus; diarrhoea viruses such as porcine epidemic diarrhoea virus; members of the Flaviviridae including flaviviruses such as yellow fever, dengue & encephalitis viruses, pestiviruses such as BVDV and unclassified viruses such as hepatitis C; members of the Bunyaviridae including phleboviruses, such as sandfly fever, nairoviruses, such as haemorrhagic fever viruses, hantaviruses, such as Hantaan and Sin Nombre, and bunyaviruses, such as bunyamwera; arenaviruses such as Lassa fever and lymphocytic choriomeningitis; astroviruses 1-5, caliciviruses such as Norwalk; members of the Reoviridae including the rotaviruses, orbiviruses and orthoreoviruses, such as bluetongue; members of the Picomaviridae including enteroviruses, such as poliovirus, ECHOvirus & coxsackievirus, rhinoviruses of all serotypes, hepatoviruses, such as hepatitis A, and aphthoviruses, such as FMDV; iridiviruses such as African seine fever virus; human and bovine papillomaviruses; filoviruses, such as Marburg and Ebola; poxviruses, such as smallpox, cowpox, variola and vaccinia; adenoviruses; orthomyxoviruses, such as influenzaviruses A,B & C and thogoto-like viruses; paramyxoviruses, such as all parainfluenzaviruses, mumps, measles, respiratory syncytial virus, Newcastle disease virus, animal distemper viruses & rinderpest. Also African swine fever virus, bovine leucosi and revirus. In one embodiment, the lyticvirus is an oncolytic virus selected from the group consisting of an adenovirus, a herpes simplex virus, a reovirus, a Newcastle disease virus, a poliovirus, a measles virus, or a vesicular stomatis virus). Additionally, the lytic virus may comprises a therapeutic gene (e.g. an apoptotic gene, a gene for tumor necrosis, a gene for starving tumor cells to death, cytolytic gene, negative I-kappa-beta, caspase, gamma globulin, h-alpha-1 antitrypsin, or E1a of adenovirus).

The term “operably linked” refers to the arrangement of various nucleic acid molecule elements relative to each such that the elements are functionally connected and are able to interact with each other. Such elements may include, without limitation, a promoter, an enhancer, a polyadenylation sequence, one or more introns and/or exons, and a coding sequence of a gene of interest to be expressed (i.e., the transgene). The nucleic acid sequence elements, when properly oriented or operably linked, act together to modulate the activity of one another, and ultimately may affect the level of expression of the transgene. By modulate is meant increasing, decreasing, or maintaining the level of activity of a particular element. The position of each element relative to other elements may be expressed in terms of the 5′ terminus and the 3′ terminus of each element, and the distance between any particular elements may be referenced by the number of intervening nucleotides, or base pairs, between the elements.

The terms “percentage of sequence identity” as used herein compares two optimally aligned sequences over a comparison window, wherein the portion of the sequence in the comparison window may comprise additions or deletions (i.e. “gaps”) as compared to a reference sequence for optimal alignment of the two sequences being compared. The percentage identity is calculated by determining the number of positions at which the identical residue occurs in both sequences to yield the number of matched positions, dividing the number of matched positions by the total number of positions in the window and multiplying the result by 100 to yield the percentage of sequence identity. Total identity is then determined as the average identity over all of the windows that cover the complete query sequence. Although not wanting to be bound by theory, computer software packages such as GAP, BESTFIT, BLASTA, FASTA and TFASTA can also be utilized to determine sequence identity.

As used herein, the term “polypeptide” encompasses both peptides and proteins, unless indicated otherwise.

A “proliferative disorder” is any cellular disorder in which the cells proliferate more rapidly than normal tissue growth. Thus a “proliferating cell” is a cell that is proliferating more rapidly than normal cells. The proliferative disorder, includes but is not limited to neoplasms. A “neoplasm” is an abnormal tissue growth, generally forming a distinct mass, that grows by cellular proliferation more rapidly than normal tissue growth. Neoplasms show partial or total lack of structural organization and functional coordination with normal tissue. These can be broadly classified into three major types. Malignant neoplasms arising from epithelial structures are called carcinomas, malignant neoplasms that originate from connective tissues such as muscle, cartilage, fat or bone are called sarcomas and malignant tumors affecting hematopoetic structures (structures pertaining to the formation of blood cells) including components of the immune system, are called leukemias and lymphomas. A tumor is the neoplastic growth of the disease cancer. As used herein, a neoplasm, also referred to as a “tumor”, is intended to encompass hematopoietic neoplasms as well as solid neoplasms. Other proliferative disorders include, but are not limited to neurofibromatosis.

The term “promoter” refers to a nucleic acid sequence that regulates, either directly or indirectly, the transcription of a corresponding nucleic acid coding sequence to which it is operably linked. The promoter may function alone to regulate transcription, or, in some cases, may act in concert with one or more other regulatory sequences such as an enhancer or silencer to regulate transcription of the transgene.

The term “promoter region” is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains (consensus sequences) responsible for the binding of RNA polymerase. Eukaryotic promoters often, but not always, contain “TATA” boxes and “CAT” boxes. Prokaryotic promoters contain Shine-Dalgamo sequences in addition to the −10 and −35 consensus sequences.

The term “propagation” as used herein refers to a productive viral infection wherein the viral genome is replicated and packaged to produce new virions, which typically can “spread” by infection of cells beyond the initially infected cell. A “propagation-defective” virus is impaired in its ability to produce a productive viral infection and spread beyond the initially infected cell.

The term “recombinant virus” is meant a virus that has been genetically altered, e.g., by the addition or insertion of a heterologous nucleic acid construct into the particle.

The term “replication” or “viral replication” as used herein, refers specifically to replication of the viral genome (i.e., making new copies of the virion DNA).

As used herein, the term “replication-competent viruses” refers to a virus capable of replication (i.e., a virus that yields progeny).

The term “replication deficient virus” as used herein refers to a virus that preferentially inhibits cell proliferation or induces apoptosis in a predetermined cell population, which supports expression of a virus replication phenotype, and which is substantially unable to inhibit cell proliferation, induce apoptos function levels characteristic of non-replicating, non-transformed cells.

The term “replication phenotype” as used herein refers to one or more of the following phenotypic characteristics of cells infected with a virus such as a replication deficient adenovirus: (1) substantial expression of late gene products, such as capsid proteins (e.g., adenoviral penton base polypeptide) or RNA transcripts initiated from viral late gene promoter(s), (2) replication of viral genomes or formation of replicative intermediates, (3) assembly of viral capsids or packaged virion particles, (4) appearance of cytopathic effect (CPE) in the infected cell, (5) completion of a viral lytic cycle, and (6) other phenotypic alterations which are typically contingent upon certain conditions for function in neoplastic cells. A replication phenotype comprises at least one of the listed phenotypic characteristics, preferably more than one of the phenotypic characteristics.

The term “substantial lysis” means at least 10% of the proliferating cells are lysed, more preferably of at least 50% and most preferably of at least 75% of the cells are lysed. The percentage of lysis can be determined for tumor cells by measuring the reduction in the size of the tumor in the mammal or the lysis of the tumor cells in vitro.

As used herein, the term “therapeutic” refers to the ability of a gene, product, protein, peptide, method and the like to alleviate at least one symptoms of a disorder, or the benefit realized from such alleviation. The term “prophylactic” refers to the ability of a gene, product, protein, peptide, method and the like to prevent or at least retard the onset of at least one symptom of a disorder, or the benefit realized from such action. As used herein, the term “enhanced therapeutic benefit” refers to the therapeutic benefit realized when more than one gene of interest is introduced to a host at the same time; the enhanced therapeutic benefit is greater than the therapeutic benefit of each of the genes administered separately. The benefit can be either additive or synergistic.

The term “therapeutically effective amount” as used herein refers to an amount of a selected DNA sequence that is sufficient to produce a therapeutic effect, e.g., inhibit metastatic tumor growth in a mammal. In one embodiment, the virus is a lytic virus. In another embodiment, a therapeutically effective amount of a lytic virus is an amount capable of producing substantial lysis of the target proliferating cells. The term “therapeutically effective amount” therefore includes, for example, an amount of such selected DNA sequence sufficient to prevent the growth of the patient's tumor, and preferably to reduce by at least 50%, and more preferably to reduce by at least 90%, the mass of a patient's tumor. The dosage ranges for the administration of the selected DNA sequence are those that produce the desired effect. Generally, the dosage will vary with the age, weight, condition, sex of the patient, type of tumor, and degree of tumor development. A person of ordinary skill in the art, given the teachings of the present specification, may readily determine suitable dosage ranges. The dosage can be adjusted by the individual physician in the event of any contraindications. In any event, the effectiveness of treatment can be determined by monitoring the extent of tumor growth and remission by methods well known to those in the field.

As used herein, the term “UTR” refers to an untranslated region sequence of mRNA. A 5′-UTR is a non-coding nucleotide sequence which lies in a viral sequence directly 5′, i.e. upstream, of the start codon of a coding gene. In the present specification, the term “region” stands for a certain range on nucleic acid (DNA or RNA). The term “5′-untranlated region of mRNA” in the present specification stands for a region that, among the mRNA synthesized by the transcription from DNA, which is present at its 5′-side and does not code for a protein.

“Vaccinia virus” refers to the virus of the orthopoxvirus genus that infects humans and produces localized lesions Vaccinia virus encodes two genes that play a role in the down regulation of PKR activity through two entirely different mechanisms. E3L gene encodes two proteins of 20 and 25 kDa that are expressed early in infection and have dsRNA binding activity that can inhibit PKR activity.

As used herein, the term “vector” or “gene delivery vector” may refer to an viral particle that functions as a gene delivery vehicle, and which comprises vDNA (i.e., the vector genome) packaged within an viral capsid. Alternatively, the term “vector” may be used to refer to the vector genome/vDNA when used as a gene delivery vehicle in the absence of the virion capsid. By “vector” is meant any genetic element, such as a plasmid, phage, transposon, cosmid, chromosome, virus, virion, etc., which is capable of replication when associated with the proper control elements and which can transfer gene sequences between cells. Thus, the term includes cloning and expression vehicles, as well as viral vectors. A vector is a replicon to which another DNA segment may be attached so as to bring about the replication of the attached segment. A “replicon” is any genetic element (e.g., plasmid, chromosome, virus) that functions as an autonomous unit of DNA replication in vivo; i.e., capable of replication under its own control. An “expression control sequence” is a DNA sequence that controls and regulates the transcription and translation of another DNA sequence. . A coding sequence is “operably linked” and “under the control” of transcriptional and translational control sequences in a cell when RNA polymerase transcribes the coding sequence into mRNA, which is then translated into the protein encoded by the coding sequence.

“Viral infection” or “virus infection” as used herein refers to infection by one or more of adenovirus, HSV, parapoxvirus orf virus, or vaccinia virus.

The present invention provides a viral vector comprising (a) a viral genome and (b) a nucleic acid sequence coding a complex 5′-UTR operably linked to the viral genome. The present invention also provides an adenoviral vector comprising (a) an adenoviral genome and (b) a nucleic acid sequence coding a complex 5′-UTR operably linked to the viral genome, wherein the DNA sequence comprises a natural or synthetic hairpin conformation with a stability of at least about ΔG≧ an absolute 50 Kcal/mol.

Unlike prior gene therapy approaches that require specific knowledge of particular cancer cells, the novel strategy targets a general characteristic that distinguishes cancer cells from normal cells, i.e., elevated eIF4E expression. This property is exploited in the present invention to repress the expression of a viral vector translationally by placing a complex 5′-UTR in front of its open reading frame. Without being bound by this theory, it is believed that cancer cells, which have higher levels of eIF4E and hence increased helicase activity, are able to continue to translate this hybrid mRNA while normal cells are not.

In one embodiment, the present invention uses the 5′-UTR of basic fibroblast growth factor (FGF-2), an angiogenic factor previously found to be translationally regulated by eIF4E. See Kevil et al., 1995. However, other complex hairpin sequences may be used for translational control by eIF4E, e.g., sequences on genes of the proto-oncogene c-myc, cyclinD1, omithine decarboxylase, or vascular endothelial growth factor (“VEGF”) . See DeBenedetti et al., 1999.

In one embodiment, the DNA sequence comprises a natural or synthetic hairpin conformation with a stability of at least about ΔG≧ an absolute 50 Kcal/mol. In one embodiment, the necessary conformation is achieved through a tight hairpin formation. In another embodiment, a relatively long palindromic oligonucleotide sequence that is self-complementary is used. See A. E. Koromilas et al., “mRNAs containing extensive secondary structure in their 5′ non-coding region translate efficiently in cells overexpressing initiation factor eIF4E,” The EMBO Journal, vol. 11, pp. 4153-4158 (1992); and A. DeBenedetti et al., 1999.

Expression of eIF4E is elevated in most solid tumors, causing translation of mRNAs that would normally be repressed by complex 5′-UTRs. In one embodiment, the present methods and compositions provide a therapeutic for solid tumors.

The viral vector system of the present invention can be used in lieu of an episome construct. The present compositions and methods allow for efficient delivery of the vector to a large number of tissues. In one embodiment, translational regulation of a lytic virus or other toxin expression is used to treat wide variety of cancers. In one embodiment, only a single injection of the vector is employed. In one embodiment, repeated injections of the vector are used to ensure greater tissue coverage, and a longer treatment for a more effective therapeutic treatment. 1001181 In one embodiment, the complex 5′-UTR used as a promoter for translation in the present invention is such that by placing this complex 5′-UTR in front of the open reading frame of the virus or conditional virus, only cells that have higher levels of eIF4E and hence increased helicase activity, are able to continue to translate this hybrid mRNA. In one embodiment, we used the 5′-UTR of basic fibroblast growth factor (FGF-2), an angiogenic factor previously found to be translationally regulated by eIF4E. See Kevil et al., 1995. However, other complex sequences could be used for translational enhancement by eIF4E, e.g., untranslated hairpin sequences on genes of the proto-oncogene c-myc, cyclin D1, ornithine decarboxylase, or vascular endothelial growth factor (“VEGF”) (otherwise known as vascular permeability factor (“VPF”)) genes. In one embodiment, the UTR sequence comprise a natural or synthetic hairpin conformation with a stability of at least about ΔG≧50 Kcal/mol.

In one embodiment, the concentration of eIF4E required to allow translation of mRNAs that would normally be repressed by the present of the complex 5′ UTRs is at least 1.5 times the concentration found in normal eukaryotic cells. In another embodiment, the concentration of eIF4E required to allow translation of mRNAs that would normally be repressed by the present of the complex 5′ UTRs is at least 2, 2.2, 2.5, 3, 3.5, 4, 4.5, 5, 6, 7, 8, 9, or 10 times the concentration found in normal eukaryotic cells.

In one embodiment, the DNA sequence comprises the 5′-UTR is a natural or synthetic conformation with a stability of at least about ΔG≦ an absolute 50 Kcal/mol. In one embodiment, the DNA sequence comprises the 5′-UTR is a natural or synthetic conformation with a stability of at least about ΔG≧ an absolute 55 Kcal/mol.

In one embodiment, the UTR sequence comprises at least 30, 40, 50, 60, 70, 80, 90, 100, 120, 150, 200, 250, 300, 400, 500, 600, 700, 800, 900, or 1000 nucleotides. In one embodiment, the UTR is an oligonucleotide sequence capable of forming a duplex. In another embodiment, the UTR is a palindromic oligonucleotide sequence capable of forming a duplex. In another embodiment, the UTR is an oligonucleotide sequence capable of forming a stable complex formation. In another embodiment, the stable complex includes loop formations.

To achieve this stability and thus a tight hairpin formation, a relatively long palindromic oligonucleotide sequence that is self-complementary is required. See A. E. Koromilas et al., “mRNAs containing extensive secondary structure in their 5′ non-coding region translate efficiently in cells overexpressing initiation factor eIF4E,” The EMBO Journal, vol.11, pp. 4153-4158 (1992); and A. DeBenedetti et al., 1999.

The free energy “ΔG” is the free energy of an oligonucleotide, which is a measurement of an oligonucleotide duplex stability. The strength (ΔG) of the resulting complexes is measured by thermal denaturation or duplex melting. The ΔG can either be expressed as a negative or a positive number depending upon whether you are looking at the stability as a measurement of free energy stored in the structure (negative) or free energy required to melt the duplex (positive).

Energy must be released overall to form a base-paired structure, and a structure's stability is determined by the amount of energy it releases. When free energy stored in the structure is a negative value, then the complex formed is in the thermodynamically stable form. Predicted enthalpy, entropy and free energy of duplex formation—the enthalpy (ΔH), entropy (ΔS), and free energy (ΔG)—are thermodynamic state functions, related by the Gibbs equation:

ΔG=ΔH-TΔS (at constant temperature and pressure)

where T is the temperature in degrees K. In practice, the enthalpy and entropy are predicted via a thermodynamic model of duplex formation and used to calculate the free energy and melting temperature.

The predicted free energy of an oligonucleotide that contains self-complementary sequences that can form intramolecular secondary structures is calculated as the most stable intramolecular structure of an oligonucleotide. “Secondary structure” refers to regions of a nucleic acid sequence that, when single stranded, have a tendency to form double-stranded hairpin structures or loops. Nucleic acids can be evaluated for their likely secondary structure by calculating the predicted ΔG of folding of each possible structure that could be formed in a particular strand of nucleic acid. Computer programs exist that can predict the secondary structure of a nucleic acid by calculating its free energy of folding. One example is the MFOLD program.

The ΔG as referred to in the specification and claims herein is given in absolute energy change value and is evident from the context by one skilled in the art. When expressed as a folded state free energy (a negative number), the more negative the ΔG (i.e., the lower the free energy), the more stable that structure is and the more likely the formation of that double-stranded structure. The stability of a secondary structure is quantified as the amount of free energy released or used by forming base pairs or the input energy required to melt such secondary structure, which in the present case would have to be ≧50 Kca/Mol. It would be obvious to one skilled in the art that the present description describes the required to melt the secondary structure since a structure having a positive free energy requires work to form a configuration and hence would be unstable and not form the required structure. Negative free energies release stored work. When quantified as the amount of free energy released or used by forming base pairs, the more negative the free energy of a structure, the more likely is formation of that structure, because more stored energy is released. For clarity's sake, the stability of the oligonucleotides of the present invention can be described as “wherein the untranslated sequence further comprises a hairpin secondary structure conformation having a stability measured as folded state free energy of ΔG≦ about −50 Kcal/Mol” instead of in terms of absolute energy change.

The protein eIF4E is the cap-binding subunit of the eIF4F complex, an ATP-dependent helicase that unwinds “excess” secondary structure in the 5′ untranslated region (UTR) of mRNAs. The low-abundance of eIF4E/F is the limiting factor for the translation of some mRNAs, particularly those with long, G/C-rich 5′-UTRs with the potential to form a stable, secondary structure. .M. J. Clemens et al., “Translational control: the cancer connection,” Int. J. Biochem. Cell Biol., vol. 31, pp. 1-23 (1999). Overexpression of eIF4E results in a specific increase in the translation of these weakly competitive mRNAs, many of which encode products that stimulate cell growth and angiogenesis, like FGF-2 and VEGF. See C. Kevil et al., “Translational enhancement of FGF-2 by eIF-4 factors, and alternate utilization of CUG and AUG codons for translation initiation,” Oncogene, vol. 11, pp. 2339-2348 (1995); C. Kevil et al., “Translational regulation of Vascular Permeability Factor by eukaryotic initiation factor 4E: Implications for tumor angiogenesis,” Int. J. Cancer, vol. 65, pp. 785-790 (1996); and P. A. E. Scott et al., “Differential expression of vascular endothelial growth factor mRNA versus protein isoforms expression in human breast cancer and relationship to eIF4E,” British. J. Cancer, vol. 77, pp. 2120-2128 (1998).

Elevating eIF4E rescues the translation of repressed mRNAs with a complex 5′-UTR, many of which encode factors required for cell proliferation, e.g., protooncogene c-myc, cyclin D1, omithine decarboxylase, fibroblast growth factor-2 (FGF-2), and vascular endothelial growth factor (“VEGF,” otherwise known as vascular permeability factor, “VPF”). See A. De Benedetti et al., “eIF4E expression in tumors: its possible role in progression of malignancies,” Int. J. of Biochemistry and Cell Biology, vol. 31, pp. 59-72 (1999).

Overexpression of eIF4E has been shown to be ubiquitous in solid tumors, including bladder, breast, cervical, colon, head and neck, and prostate, as well as in many malignant cell lines. See J. P. Crew et al., Eukaryotic initiation factor-4E in superficial and muscle invasive bladder cancer and its correlation with vascular endothelial growth factor expression and tumour progression,” Br. J. Cancer, vol. 82, pp. 161-166 (2000); V. V. Kerekatte et al., “The proto-oncogene/ translation initiation factor eIF4E: a survey of its expression in breast carcinomas,” Int. J. Cancer., vol. 64, pp. 27-31 (1995); I. B. Rosenwald et al., “Upregulation of protein synthesis initiation factor eIF4E is an early event during colon carcinogenesis,” Oncogene, vol. 18, pp. 2507-2517 (1999); C. O. Nathan et al., “Detection of the proto-oncogene eIF4E in surgical margins may predict recurrence in head and neck cancer,” Oncogene, vol.15, pp. 579-584 (1997); Y. Miyagi et al., “Elevated levels of eukaryotic initiation factor eIF4E mRNA in a broad spectrum of transformed cell lines,” Cancer Letters, vol. 91, pp. 247-252 (1995); B. Anthony et al., “Overexpression of the protooncogene-translation factor eIF4E in breast carcinoma cell lines,” Int. J. Cancer, vol.65, pp. 858-863 (1996); and I. B. Rosenwald, “Upregulated expression of the genes encoding translation initiation factors eIF4E and eIF-2alpha in transformed cells,” Cancer Letters, vol. 102, pp. 113-23 (1996).

The present invention provides for a novel gene therapy for cancer, which unlike most prior approaches, does not require specific knowledge of the cancer cells, but instead targets a general characteristic that distinguishes cancer cells from normal cells, i.e., elevated eIF4E expression.

The specific functional characteristics of the claimed sequences are an adequate representation of the genus and that the sequences that fall within the scope of the present claims are easily ascertained by any person of skill in the field of molecular biology and medical science and that such person would know how to sequence various DNA sequences as well as how to test any particular sequence in order to determine whether or not the sequence inhibits translation of the viral sequence in the absence of eIF4E and allows translation of the viral sequence into a virus in the presence of eIF4E.

It is well within the ability of person in the field of molecular biology and medical science to determine the appropriate sequences of the present invention given that: (1) the amount of testing required is relatively small especially since most of the work can be done with tissue culture experiments as the proof of principle with the animal studies was already provided; (2) testing of any particular sequence in question would not require direction or guidance beyond that known in the art; (3) the current state of knowledge in the art and relative skill of those in the art is quite high; (4) well-known procedures exist for sequencing various DNA sequences capable of producing a messenger RNA sequences that comprises an untranslated palindromic sequences; and (5) determining whether or not a sequence falls within the scope of the claims is quite straightforward since all of the materials and methods that would be required to determine if a particular untranslated sequence inhibits translation of the viral sequence in the absence of eIF4E and allows translation of the viral sequence into a virus in the presence of eIF4E are quite routine in the art.

In one embodiment, the present invention provides conditionally replicative adenoviruses (CRAds) as therapeutic agents applied to cancer treatment. In one embodiment, cancer-specific replication of CRAds result in viral-mediated oncolysis of infected tumor tissues and release of the virus progeny, capable of further propagating in surrounding tumor cells but not in those of normal tissues, which would be refractory to CRAd replication.

Gene Therapy Approaches to Cancer

Studies of the molecular mechanisms underlying neoplastic transformation and progression have resulted in the understanding that cancer is a genetic disease, deriving from the accumulation of a series of acquired genetic lesions. Despite advances in chemotherapy, radiation delivery and surgical treatment regimens, survival from many advanced cancers remain poor, and it is apparent that alternative treatment approaches are necessary. Therefore, gene therapy/virotherapy remains a promising strategy for the treatment of cancer. The existing approaches to gene therapy/virotherapy of cancer can be divided into five broad categories: 1) mutation compensation, 2) molecular chemotherapy, 3) genetic immunopotentiation, 4) genetic modulation of resistance/sensitivity and 5) oncolytic therapy or virotherapy. Any of the above gene therapy/virotherapy approaches is fundamentally based on the ability of vector to deliver the therapeutic gene or replication-competent viral genome to target cells with a requisite level of efficiency. A number of characteristics of the Adenovirus type 5 (AdS), including capacity for highly efficient in vivo gene delivery, make it an optimal gene therapy/virotherapy vector suitable for a large number of gene therapy/virotherapy approaches and set Ad5 apart from other vector choices.

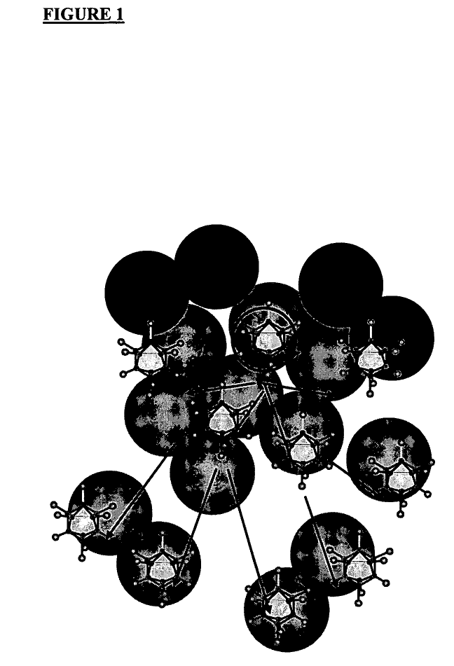

Using conditionally replicative adenoviruses represent a method to achieve efficient tumor cell oncolysis and mitigate tumor cell infection limitations. Despite these advantages, overall efficacy of Ad-based cancer gene therapy/virotherapy approaches remains limited by sub-optimal vector delivery efficiency in cancer tissues. Of note, human trials carried out to date have demonstrated relatively inefficient gene transfer to tumor cells in contexts whereby non-replicative adenoviruses have been employed using in vivo delivery schemas. Thus, the requirement for quantitative in vivo tumor transduction is the key issue that has to be addressed for further development of the cancer gene therapy/virotherapy field. Thus, it is clear that augmenting the gene transfer efficacy of adenoviruses for cancer targets is an important tool for deriving their full benefit. However, transductional targeting alone does not allow sufficient cancer selectivity for adenoviruses, due to lack of known cell surface receptors (cell surface markers) highly specific for cancer cells that are not also present on normal cells. Another fact that has limited effective tumor cell transduction in the context of cancer gene therapy/virotherapy approaches is that the adenoviruses employed have been rendered replication-incompetent. In this case, tumor cell infection is a terminal event, whereby post-infection viral replication (and consequential amplification) does not occur. One conceptual approach to achieve an amplification effect is via selective replication of the delivered viral vector post-infection such that lateral spread of the progeny vector may occur. In this approach, a conditionally replication-competent virus replicates in transduced tumor cells and not in normal tissue. Production of virus progeny from transduced tumor cells would then allow infection of the neighboring tumor cells (FIG. 1). Additionally, utilization of viruses, which replicate through out a lytic cycle, would result in viral-mediated oncolysis, a process that is of therapeutic utility. In support of this conceptual approach, replicative viral systems have been utilized as novel anti-tumor therapies. Wild-type or attenuated viruses with the ability to replicate within specific tissues have been used in human clinical trials to achieve specific oncolysis of various neoplasms. Those viruses include: adenovirus [4], mumps virus [5,6], and West Nile virus [7]. More recently, the parvovirus H-1 [8,9] and the herpes simplex virus [10,11] have also been shown to achieve viral oncolysis of tumor cells.

Adenoviruses Possess Unique Attributes Fundamental for Development as Conditionally Replicating Anti-Tumor Agents

One of the beneficial attributes of the Ad vector as an anti-tumor therapy agent is that this virus possesses a lytic life cycle, which can be exploited for oncolysis. In this context, Bischoffet al. [12], have recently employed Conditionally Replicative Adenovirus (CRAd) defective in early gene regions to accomplish tumor-specific oncolysis. Specifically, dl1520 (ONYX-015) is an E1B-55 kDa gene deleted CRAd that has been demonstrated to replicate in and selectively destroy tumor cells such as ovarian cancer, which lack p53 function [12]. Indeed, significant anti-tumor activity was demonstrated with ONYX-015 both in vitro and in intraperitoneally treated murine models of ovarian cancer [13.14]. ONYX-015, has undergone extensive testing in the clinic, and has proven safe with promising signs of efficacy [4,15]. This experience thus established the concept that CRAd systems can accomplish a significant anti-tumor effect. A more detailed knowledge of the Ad replication cycle should make it feasible to develop advanced CRAds that achieve therapeutic oncolysis over-and-above that achieved with the first generation ONYX-015 CRAd system.

S strategy to Render CRAds Replicating Selective, Based on Targeting E1A Transcription to Cancer Cells

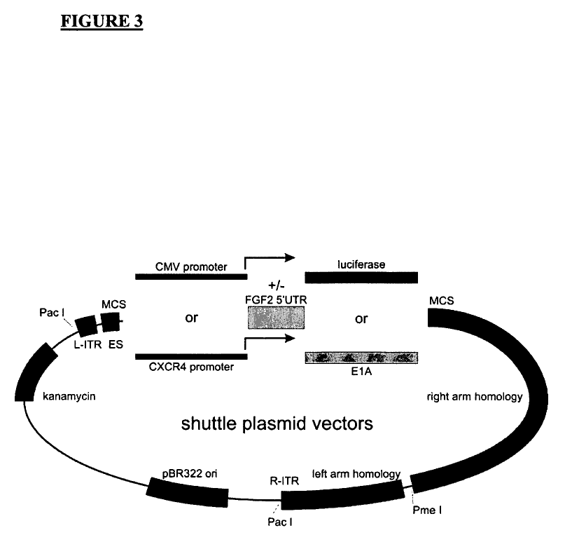

For both replicative and non-replicative vector-based approaches, ectopic infection of non-tumor targets can elicit tumor-associated toxicities. This issue has been addressed by means of “targeting” the vector exclusively to tumor cells. Methods to this end have attempted to alter viral tropism by fiber genetic modification or by means of restricting a therapeutic gene expression via a tumor/tissue selective promoter (TSP) to achieve targeting, either of the delivered therapeutic gene in a non-replicative vector context, or viral replication/oncolysis in a CRAd context. The most widely used method to create an Ad-based vector for targeted expression of transgene or a CRAd is to utilize a TSP for transcription of Ad5 E1 gene. As result of this approach, CRAds replicate only in cells with high activity of the promoter, which allow expression of E1 at the levels sufficient to mediate viral replication. Thus, the key issue in this strategy is specificity of the tumor/tissue selective promoter in the context of the Ad genome. Furthermore, low activity in the liver is critical for avoiding adverse effects, as the majority of virus released into the blood stream localizes to the liver. It is noteworthy, however, that many promoters that exhibit specificity in plasmid-based constructs do not show such specificity in adenoviruses [16]. We have thus sought promoters usable for CRAd construction with these considerations in mind. In this application we propose to use promoter of CXCR4 gene, a receptor for stromal derived factor-1 (SDF-1), a member of the CXC-group of small chemo attractive cytokines (chemokines). Expression of CXCR4 was found markedly up regulated in breast cancer cells but undetectable in normal mammary epithelial cells [17]. In addition expression of CXCR4 has been associated with many other types of cancer such as prostate cancer, myeloma and lymphoma, and has been implicated in tumor progression and metastasis [18-20]. Recently the CXCR4 promoter was used to drive an efficient reporter gene expression in the context of Ad5 based vectors [21]. In addition, as demonstrated by our preliminary data, a reporter gene driven by the CXCR4 promoter in the context of replication-deficient Ad vector shows the highest expression level among other TSPs in several HNSCC cell lines. Moreover, the CXCR4 promoter is even more active than CMV promoter in the same Ad vector context (FIG. 3). Therefore, CXCR4 promoter activity should be high enough to drive efficient expression of E1 genes in the CRAd context. Altogether, these data suggest that the CXCR4 gene promoter is a suitable TSP for construction of novel tumor-selective transcriptionally targeted CRAds. It has been recently shown that in contrast to cancer cells activity of CXCR4 promoter in normal tissues such as liver is rather low. Nonetheless some residual (background) activity of the promoter can still be detected in human liver slices [Zhu et al., personal communication]. This circumstance necessitates the further improvement of the proposed CRAd to enhance its cancer specificity. We reasoned that the cancer control of E1A gene expression mediated by the CXCR4 gene promoter could be secured by imposing another level of cancer targeting on the E1A gene expression. This would potentially reduce or even abolish the promoter leakage in the liver and other normal tissues. We hypothesize that one way to additionally restrict the CXCR4-regulated CRAd replication to cancer cells is to add cancer-specific translational control. The principal of such a control and rationale are described below.

Improvement of the Adenovirus Vector Based on Targeting of E1A mRNA Translation to Cancer Cells: Regulation of Protein Synthesis for Restriction of Transgene Expression to Cancer Cells

Protein synthesis is energetically the most expensive process in the cell, and not surprisingly, translation rates are tightly regulated [22]. In mammals, most of the regulation operates at the level of translation initiation, rather than elongation or termination [23,24]. The initiation process is comprised of three steps: 1) formation of the 43S complex, composed of a 40S ribosomal subunit and the initiation factors eIF-2, eIF-3, Met-tRNAᵢ and GTP; 2) formation of the 48S complex containing mRNA; and 3) joining of the 60S subunit to form the complete 80S complex. In most circumstances, the second step is rate limiting and hence, subject to regulation. This step is also a point of discrimination, since one particular mRNA is selected from the untranslated pool of messages and recruited to the ribosomes. This process is mediated by the eIF-4 group of factors, of which, eIF4E is the least abundant and rate limiting [25-27] The importance of protein synthesis in growth regulation and the role of eIF4E in this process was confirmed by the observation that overexpression of eIF4E causes malignant transformation of cells in culture [28,29].

The Role of eIF4E in Protein Synthesis

The synthesis of each protein ultimately depends on the relative abundance of its mRNA and its intrinsic translatability, i.e., the capacity of that particular mRNA to interact with components of the translation initiation machinery. This property of the translation initiation process establishes an order of priorities among the different mRNAs to be translated. Such a hierarchy in protein synthesis is extremely important for gene expression. In eukaryotes, the flow of information from genes to proteins is too slow to accommodate rapid changes in the environment. Eukaryotes compensate for this problem by maintaining a pool of mRNAs that are not immediately utilized. Some of these “translationally repressed” mRNAs may, for instance, encode growth factors which can be rapidly produced under conditions that require cells to re-enter rapid division [30,31]. A theoretical treatment of mRNA competition for translation [32] identified two types of structurally different mRNAs: weak and strong. Weak mRNAs, about 5-10% of the total, are translationally repressed in quiescent cells; weak and strong mRNAs are translated in rapidly proliferating cells [33,34] and in cancerous cells in particular, due to a change in the translation initiation capacity. The eIF4E specifically binds to the 7-methylguanosine-containing cap of the mRNA in the first step of mRNA recruitment for translation [35]. The eIF4E is also a subunit of the eIF-4F complex, a helicase that unwinds the secondary structure at the 5′-UTR of mRNA. This latter function is critical during “scanning” for exposing and locating the translation start site [36-40]. The low abundance of eIF4E creates a situation of competition among different mRNA species, such that mRNAs with long and highly structured 5′-UTRs (weak) are out competed for binding to ribosomes by the strong mRNAs [41]. An analysis of sequence data from 700 vertebrate mRNAs has shown that more than 90% contain 5′-UTRs that are less than 200 nucleotides long and devoid of upstream AUGs, which is characteristic of strong mRNAs [42]. Weak mRNAs contain long GC-rich 5′-UTRs, with potential for forming stable secondary structure, and/or upstream AUGs [43-45]. Many such mRNAs code for oncoproteins, cell cycle regulators, growth factors and their receptors. Elevating the level or activity of eIF4E results in a large and selective increase in the translation of weak mRNAs [30].

The Role of eIF4E in Neovascularization

Translational control exerted by low eIF4E concentration is critical for the appropriate repression of gene expression needed to maintain organ/tissue differentiation. Conversely, there seems to be an obvious need for a mechanism that is based largely on altered translational efficiency to respond quickly to pathological emergencies. One clear-cut example for this concept is the process of neovascularization. This process is the perennial capacity of the organism to provide new capillary sprouts to tissues in need. This reflects both physiological situations, such as growth of endometrial tissue during the reproductive cycle or placental development, and pathological conditions like ischemialhypoxia, vascular retinopathy, and tumor angiogenesis [46]. The process of neovascularization is complex and involves many steps and cell types [47]. The entire process needs to be remarkably fast to minimize blood and fluid loss. Rapid responses to injury, inflammation, or tumor angiogenesis would be impaired if cells were to rely entirely on de novo gene expression. Instead, a mechanism that is based largely on altered translational control (eIF4E activity) has evolved to respond quickly to the imperative of achieving adequate oxygenation [30]. Elevated eIF4E results in drastically increased synthesis of basic fibroblast growth factor (FGF2) [48,31] and vascular endothelial growth factor (VEGF) [49], both of which are encoded by mRNAs with long and complex 5′-UTRs. Indeed, there is a strong correlation between the expression of eIF4E and VEGF in tumor biopsies [50]. FGF2 and VEGF are the two most powerful mitogens for vascular endothelia and are essential for tumor vascularization [51]. The up-regulation of eIF4E could also be a pre-requisite for establishing greater protein synthesis outputs, possibly a necessary development for cancer cells to sustain their rapid proliferation.

eIF4E in Cancer and as a Target for Gene Therapy/Virotherapy

We now know that overexpression of eIF4E is ubiquitous in solid tumors and malignant cell lines [52-55]. Upregulation of eIF4E appears to be a pre-requisite for vascularization of the primary tumor [52]. Reduction of eIF4E with antisense RNA in a breast carcinoma line crippled its angiogenic and tumorigenic properties, along with the capacity to synthesize FGF2 [31]. It is important to emphasize that elevated eIF4E does not always correlate with rapid cell division. Even cells from the intestinal mucosa, which have rapid turnover rates, do not have high levels of eIF4E, although colon carcinomas do [53]. This, of course, does not exclude the possibility that specific cells or local areas in a tissue may express higher levels of eIF4E, but in general eIF4E elevation is a characteristic of malignant cells, not of normally dividing cells. Likewise, there are clearly specific tissues that express FGF2, and as such, must be able to translate the FGF2 mRNA. However, cells that overexpress eIF4E typically have a 50 to 100-fold higher rate of FGF2 synthesis than normal cells without a corresponding increase in its mRNA. Therefore, they specifically have a greater capacity to translate the FGF2 mRNA [48]. Recognizing the nearly ubiquitous eIF4E overexpression in solid tumors, we devised a strategy that targets selectively cancer cells. Since elevated eIF4E specifically facilitates the translation of mRNAs with long and structured 5′-UTR, one can design mRNAs that will be selectively translated in cancer cells. Based on these finding, we have previously devised a strategy for targeting suicide gene expression to tumor cells based on restricting transgene translation to eIF4E overexpressing cells. Specifically, we constructed an expression vector in which the herpes simplex virus thymidine kinase (HSV-Tk) cDNA was preceded by the long 5′-UTR of FGF2. We hypothesized that this construct would be a more selective target to cancer cells, and hence, sparing the population of normal cells (including the bone marrow) even if such cells should become transduced. This work now published [2,3] showed that translational regulation of therapeutic genes can be achieved with in vitro and in vivo models, and furthermore translational regulation allows strong discrimination between cancer and normal cells (due to elevated levels of eIF4E).

Combining Both Transcriptional and Translational Targeting to Improve Cancer Specificity of CRAds

In order to generate an effective oncolytic CRAd agent, its replication needs to be rendered a) highly cancer selective and b) highly efficient in a particular type of cancer tissue/cell. Such selectivity and efficiency of replication can be achieved by controlling the expression of Ad5 E1 genes, essential for Ad replication function, at two levels: transcriptional and translational. Transcriptional targeting allows efficient transcription of the E1 genes and thus replication of CRAds only in certain tissue or cancer cells (tumor) but not in normal cells (tissues).

Transcription of the genes responsible for replication can be modulated by placing them under control of various tissue- or tumor-specific promoters (TSPs)

Apart from cancer specificity in vivo transcriptional activity of TSP needs to be high enough to provide sufficient levels of E1 gene expression, required to allow CRAd replication. Given the limited number of available TSPs with cancer induction profiles it is difficult to create an optimal CRAd agent for most types of cancers. In fact, most of the promoters characterized so far possess tissue rather than cancer specificity. Even transcriptional targeting by cancer-specific promoters is never absolutely specific and may result in a significant level of background (non-specific) activity in normal tissues and organs, particularly liver. Unfortunately, translational targeting is not absolutely specific either, since eIF4E can be up regulated not only in tumors but also in certain types of normal tissues. Since the effect of translational alleviation of the FGF25′-UTR-containing mRNAs depends on the levels of eIF4E, the translational targeting approach alone may not provide sufficient degree of cancer specificity when used in the context of a CRAd. Therefore, it is highly important to create a CRAd that combines both transcriptional and translational cancer targeting to ensure efficacy and specificity of the virus in vivo. We have tested this novel type of dual-level cancer targeting, which combines regulation of E1A gene expression at both transcriptional and translational levels. This approach takes advantage of cancer specificity of the CXCR4 gene promoter on the one hand and translational features of E1A mRNA placed under translational control of FGF2 mRNA 5′-UTR, on the other.

SUMMARY

We have validated of the principal of dual-level cancer targeting that combines regulation of E1A gene expression at both transcriptional and translational levels, in the development of a cancer-specific CRAd for the head and neck group of cancers.

After validation of the principal of dual-level cancer targeting of reporter gene expression in head and neck cancer as a model, we propose to use it in the development of a cancer-specific CRAd for the head and neck group of cancers. Importantly, this CRAd may potentially be suitable for other types of cancers that support CXCR4 promoter activity and over-express eIF4E. In one embodiment, the present invention provides for dual-level of cancer targeting to overcome any possible leakage of the CXCR4 promoter in normal tissues. In another embodiment, the dual-level cancer targeting is further combined with infectivity enhancement/transductional re-targeting of CRAds to Ad3 receptor achieved by serotype chimerism technology of fiber modification developed previously [56-58].

Supportive Data

We constructed a replication-defective Ad5-based vector with the E1A promoter and coding regions replaced by a cassette encompassing the luciferase reporter gene placed under transcriptional control of the CXCR4 gene promoter. As shown in FIG. 3, the luciferase reporter gene coding sequence was used either with or without FGF2 mRNA 5′-untranslated region (5′-UTR) sequence inserted upstream of the open reading frame (ORF). Likewise, the Ad5 E1A gene coding sequence was used either with or without FGF2 mRNA 5′-untranslated region (5′-UTR) sequence inserted upstream of the ORF. Replacement of E1A region in the pVK400 rescue vector, which was used to generate the Ad5 backbone, was accomplished in the E. coli recombination competent strain BJ-5183 by homologous recombination. To this end the bacterial strain was co-transformed (by electroporation) with the rescue vector linearized at a unique Cla I site engineered in the E1 region of the Ad5 genome and linearized shuttle vectors containing a reporter gene expression cassettes with or without FGF25′-UTR in front of the reporter gene sequences. The cassettes in the shuttle vectors are flanked by Ad5 sequences located right upstream and downstream of the E1 region (right and left arm homology sequences) in the Ad5 genome and used to insert the shuttle vector cassette in place of E1 by homologous recombination. Since the genomes of such recombinant Ad vectors derived from pVK400 contain the wild-type fiber gene, no additional homologous recombination steps were necessary to create replication defective recombinant Ad vectors expressing the reporter or E1A genes.

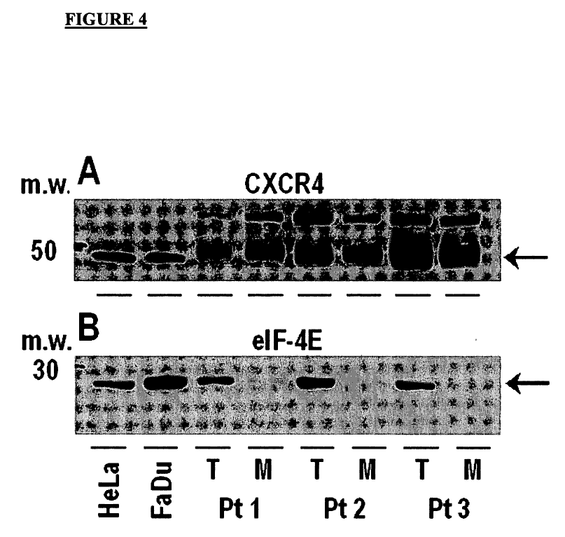

In the data shown in FIG. 4., endogenous expression of eIF4E was compared to CXCR4 in patient HNSCC tumor samples. FIG. 4 shows three tumor samples, T1, T2, and T3 and T2, and the matched adjacent histologically normal mucosa, M1, M2, and M3, obtained from surgical resection margins. All three tumor samples (T) overexpressed eIF4E; of the three margins (M) shown, two margins were negative for eIF4E, while one (M1) expressed eIF4E at low but detectable levels. CXCR4 levels were high in all tumor samples and margins. Although CXCR4 was detected in both tumor and matched normal tissues, Western blot analysis showed strong increased expression of eIF4E in tumors compared with adjacent mucosa. These results underscore the need for multiple levels of regulated expression to protect normal cells from non-specific killing by virotherapy agents.

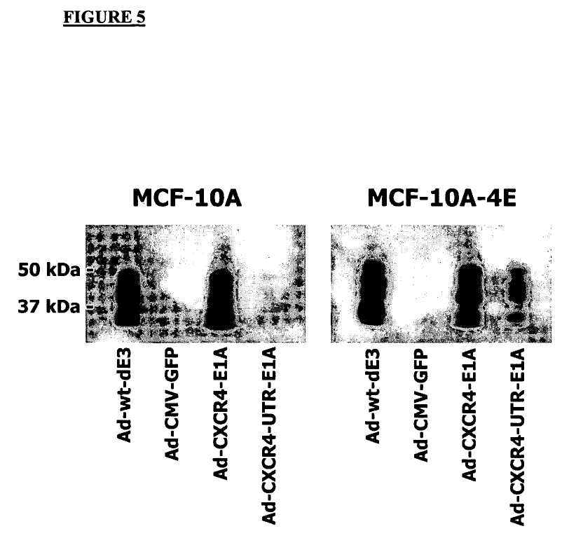

To determine if the differential synthesis of E1A protein from the UTR-E1A mRNA was largely a consequence of the level of eIF4E protein translation, we used Western blot analysis to determine protein levels. First, the cell lines were infected with Ad-CXCR4-E1A or with Ad-CXCR4-UTR-E1A, and after 48 hours, the levels of E1A protein levels were determined by Western blot analysis. As a positive control, cells were infected with Ad-wt-dE3, and as a negative control, cells were infected with Ad-CMV-GFP. As shown in FIG. 5, as anticipated in a normal breast epithelial cell line that express low levels of eIF4E (MCF-10A), the E1A protein expression level was significantly lower after infection with Ad-CXCR4-UTR-E1A compared to infection with Ad-CXCR4-E1A. Importantly, in a breast epithelial cell lines (MCF-10A-4E), which has higher levels of eIF4E, E1A protein expression levels were significantly higher after infection with Ad-CXCR4-UTR-E1A compared to infection in MCF-10A cells. This result serves to confirm our hypothesis that the expression of E1A mRNA in the context of the Ad-CXCR4-E1A vector is independent of eIF4E protein levels. By contrast, the translation of the E1A mRNA in the context of the Ad-CXCR4-UTR-E1A vector is strongly dependent on the presence of elevated levels of eIF4E, such as occurs in a variety of cancers.

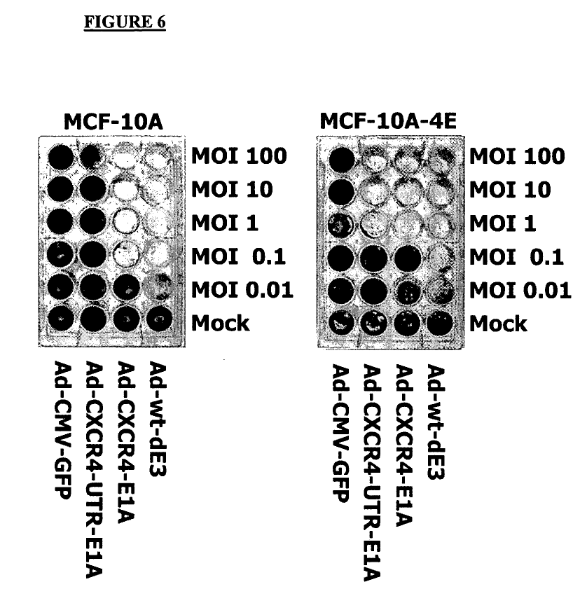

To confirm that the differential synthesis of E1A protein from the UTR-E1A mRNA results in a differential oncolytic effect of the CRAd corresponding to the level of eIF4E protein translation, we used a crystal violet staining assay to determine cell oncolysis (FIG. 6.). First, the cell lines were infected with Ad-CXCR4-E1A or with Ad-CXCR4-UTR-E1A, and after 48 hours, the levels of E1A protein levels were determined by Western blot analysis. As a positive control, cells were infected with Ad-wt-dE3, and as a negative control, cells were infected with Ad-CMV-GFP. As anticipated, in a normal breast epithelial cell line that express low levels of eIF4E (MCF-10A), the oncolytic activity was significantly lower after infection with Ad-CXCR4-UTR-E1A compared to infection with Ad-CXCR4-E1A. Importantly, in a breast epithelial cell lines (MCF-10A-4E), which has higher levels of eIF4E, oncolytic activity after infection with Ad-CXCR4-UTR-E1A was significantly higher that in MCF-10A cells, and was comparable to compared to oncolytic activity after infection with Ad-CXCR4-URT-E1A. This result serves to confirm our hypothesis that the expression of E1A mRNA in the context of the Ad-CXCR4-E1A vector is independent of eIF4E protein levels.

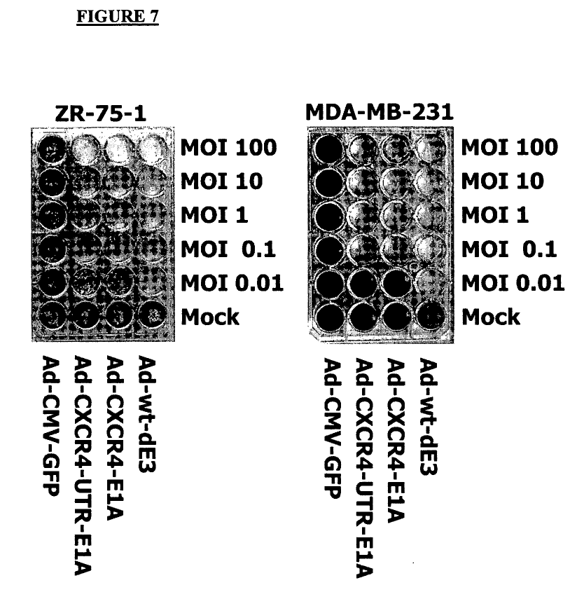

The cell killing capacity of two different CRAd agents was compared to wild-type Ad5 (Ad-wt-dE3) in two different breast cancer cell lines. Each of the two CRAd agents tested used the CXCR4 tumor specific promoter to regulate expression of the adenovirus E1A. However, while one construct expressed a wild-type E1A transcript (Ad-CXCR4-E1A), a second construct expressed the 5′-UTR of FGF2 inserted upstream of the E1A open reading frame (Ad-CXCR4-UTR-E1A). For this experiment, we infected the cells with increasing doses (from 0.01 ifu/cell to 100 ifu/cell) of each virus, allowed multiple cycles of viral replication over the following days and the remaining the live cells attached to the wells were stained with crystal violet on day 10. As shown in FIG. 7, in each of the two breast cancer cell lines, the CRAd that expressed the 5′-UTR of FGF2 inserted upstream of the E1A open reading frame (Ad-CXCR4-UTR-E1A) showed a similar cell killing effect compared to the CRAd that expressed a wild-type E1A transcript (Ad-CXCR4-E1A).

Data Summary

These results clearly demonstrate the feasibility of combining a cancer-specific targeting approach using tissue- or tumor-specific promoters, together with another cancer-specific regulation at the level of protein translation.

Adenoviral Genome

During the past decade utilization of transcriptional control elements (promoters) with cancer-specific induction profiles for expression of adenoviral E1A and E1B genes essential for replication of the virus has led to the development of next generation CRAds that are transcriptionally targeted to cancer cells. Therefore, choosing an appropriate tumor-specific promoter (TSP) for a given type of cancer is critical to achieve high cancer specificity of CRAds. Unfortunately, the number of available promoters with cancer-specific activation properties is very limited. In addition, for many cancer tissues optimization of such promoter activity may represent a major problem and significantly affect cancer specificity of the targeted conditionally replicative adenovirus. Given that adenovirus type 5 (Ad5)-based conditionally replicative adenoviruses have a natural liver tropism, any background activity of cancer-specific promoters in normal tissues (and subsequent liver toxicity) presents a particularly critical problem that warrants development of novel approaches to improve cancer specificity of CRAds.

Adenoviral Genome

Any subtype mixture of subtypes, or chimeric adenovirus can be used as the source of the viral genome for generation of an adenoviral vector in conjunction with the present invention. Preferably the genome of a human serotype adenovirus is used, such as a type 2 (Ad2) or type 5 (Ad5) adenoviral genome. Although any suitable adenoviral genome can be used in conjunction with the present invention, the Ad5 adenoviral genome is most preferred, and the present invention is described further herein with respect to the Ad5 serotype.

In one embodiment, the adenoviral genome used in conjunction with the present invention is replication deficient in addition to conditionally replicative in a cell with conditions with an overexpression of eIF4E. This would provide a second level of defense against undesirable replication in non-target cells. A deficiency in a gene, gene function, or gene or genomic region, as used herein, is defined as a deletion of sufficient genetic material of the viral genome to impair or obliterate the function of the gene whose nucleic acid sequence was deleted in whole or in part and to provide room in, or capacity of, the viral genome for the insertion of a nucleic acid sequence that is foreign to the viral genome. Such a deficiency can be in a gene or genome region essential or unessential for propagation of the adenoviral vector in a non-complementing cellular host. A deficiency in an adendviral genome region essential for such propagation (e.g., early region 1 (E1), early region 2A (E2A), early region 2B (E2B), early region 4 (E4), late region 1 (L1), late region 2 (L2), late region 3 (L3), late region 4 (L4), and late region 5 (L5)) renders an adenoviral vector based on that adenoviral genome replication deficient.

The adenoviral vector of the present invention may be multiply replication deficient, i.e., it is deficient in at least two genome regions required for viral propagation in a non-complementing cellular host (i.e., viral replication in vitro. Such regions include the E1, E2, E4, or L1-L5 regions. Even though the E1 region can be considered as consisting of early region 1A (E1A) and early region 1B (E1B), a deficiency in either or both of the E1A and/or E1B regions is considered as a single deficiency in the context of the present invention. In addition, such a vector can be deficient in one or more regions that are not required for viral propagation, e.g., the vectors can be additionally deficient in early region 3 (E3).

The present invention is not limited to adenoviral vectors that are deficient in gene functions only in the early region of the genome. Also included are adenoviral vectors that are deficient in the early and late regions of the genome, as well as vectors in which essentially the entire genome has been removed, in which case it is preferred that at least either the viral ITRs and some of the promoters or the viral ITRs and a packaging signal are left intact.

In another embodiment, the vector comprises at least one expression cassette which includes (i.e., comprises) a nucleic acid sequence coding for a toxin. A nucleic acid sequence coding for a toxin is described in detail in U.S. Pat. No. 6,759,394.

Preferably, the nucleic acid sequence encoding a toxin further comprises a transcription-terminating region such as a polyadenylation sequence. Any suitable polyadenylation sequence can be used, including a synthetic optimized sequence, as well as the polyadenylation sequence of BGH (Bovine Growth Hormone), polyoma virus, TK (Thymidine Kinase), EBV (Epstein Barr Virus), and the papillomaviruses, including human papillomaviruses and BPV (Bovine Papilloma Virus). A preferred polyadenylation sequence is the SV40 (Human Sarcoma Virus-40) polyadenylation sequence.

Preferably, the nucleic acid sequence encoding a toxin is operably linked to (i.e., under the transcriptional control of) one or more promoter and/or enhancer elements, for example, as part of a promoter variable expression cassette. Techniques for operably linking sequences together are well known in the art. Any suitable promoter or enhancer sequence can be used in conjunction with the present invention. Suitable promoters and enhancer sequences are generally known in the art.

Vector Construction

The present invention provides compositions and methods of producing a conditionally replicative adenoviral vector comprising (a) providing an adenoviral genome, (b) inserting a nucleic acid sequence DNA sequence comprising a promoter operatively linked to a transcription sequence; wherein the transcription sequence, when transcribed, produces a messenger RNA sequence that comprises a translatable sequence encoding a viral particle, and an untranslated sequence; wherein the untranslated sequence inhibits translation of the viral sequence under conditions that exist within normal mammalian cells that do not overexpress eukaryotic initiation factor eIF4E; and wherein the untranslated sequence allows translation of the toxin sequence under conditions that exist within mammalian cells that overexpress eukaryotic initiation factor eIF4E relative to normal cells.

In one embodiment, the untranslated sequence further comprises a hairpin secondary structure conformation having a stability measured as folded state free energy of ΔG≦ about −50 Kcal/Mol.

In another embodiment, the present invention provides for a replication competent adenovirus-free stock of the viral vector of the present invention. In another embodiment, the present invention provides for a pharmaceutical composition comprising the viral vector of the present invention and a pharmaceutically acceptable carrier, wherein the pharmaceutical composition does not contain replication-competent adenoviruses. In another embodiment, the present invention provides for a host cell comprising the viral vector of the present invention. In another embodiment, the present invention provides for a method of treating a tumor or cancer in a mammal comprising administering an anti-tumor or anti-cancer effective amount of the conditionally replicative viral vector of the present invention directly to the tumor or cancer of the mammal. In another embodiment, the an anti-tumor effective amount of the viral vector is administered to a tumor in a mammal.

In another embodiment, the present invention provides for a method of treating a cell proliferation disease comprising administering to a subject in need of such treatment a therapeutically-effective amount of the compositions of the present invention.

In another embodiment, the administering is in an amount effective to inhibit cell growth. In another embodiment, the DNA sequence is administered by administering an expression vector encoding the DNA sequence to cells. In another embodiment, the expression vector is delivered within a liposomal construct. In another embodiment, the the expression vector is delivered within a host cell. In another embodiment, the expression vector is a viral vector.

In another embodiment, the untranslated sequence allows translation of the viral sequence under conditions that exist within mammalian cells that overexpress eukaryotic initiation factor eIF4E at least 2-fold greater relative to normal cells.

In another embodiment, the vector further comprises a sequence encoding for a toxin. In another embodiment, the encoded toxin is a conditional toxin. In another embodiment, the encoded conditional toxin is a herpes thymidine kinase.

In another embodiment, the present invention provides for a method for selectively expressing a viral particle within a cell comprising administering to the cell a messenger RNA sequence that comprises a translatable sequence encoding a virus, and an untranslated sequence; wherein the untranslated sequence inhibits translation of the viral sequence under conditions that exist within normal mammalian cells that do not overexpress eukaryotic initiation factor eIF4E and wherein the untranslated sequence allows translation of the viral sequence under conditions that exist within mammalian cells that overexpress eukaryotic initiation factor eIF4E relative to normal cells. In another embodiment, the untranslated sequence comprises an mRNA sequence with a secondary structure conformation having a stability measured as folded state free energy of ΔG≦ about −50 Kcal/Mol. In another embodiment, the administering is in an amount effective to inhibit cell growth. In another embodiment, the messenger RNA sequence is administered by administering an expression vector encoding the messenger RNA sequence. In another embodiment, the viral vector is delivered within a host cell.

In another embodiment, the present invention provides for a method of treatment for cancer in a mammal, comprising administering to a mammal in need of such treatment a therapeutically effective amount of a DNA sequence comprising a promoter operatively linked to a transcription sequence; wherein the transcription sequence, when transcribed, produces a messenger RNA sequence that comprises a translatable sequence encoding a virus, and an untranslated sequence; wherein the untranslated sequence inhibits translation of the viral sequence under conditions that exist within normal mammalian cells that do not overexpress eukaryotic initiation factor eIF4E; and wherein the untranslated sequence allows translation of the viral sequence under conditions that exist within tumor cells that overexpress eukaryotic initiation factor eIF4E; relative to normal cells.

In another embodiment, the untranslated sequence allows translation of the viral sequence under conditions that exist within tumor cells that overexpress eukaryotic initiation factor eIF4E at least 2-fold greater relative to normal cells. In another embodiment, the untranslated sequence allows translation of the viral sequence within tumor cells in which the presence of eukaryotic initiation factor eIF4E allows the translation of the virus, the virus is translated to kill the tumor cells. In another embodiment, the the majority of non-tumor cells in the mammal are not killed due to the low levels of eukaryotic initiation factor eIF4E typically present in non-tumor cells. In another embodiment, the cancer is a metastatic tumor. In another embodiment, the cancer is a solid tumor. In another embodiment, the metastatic tumor is associated with a mammalian cancer selected from the group consisting of bladder, breast, cervical, colon, lung, prostate, and head and neck.

The present invention also provides a method of producing an adenoviral vector comprising (a) providing an adenoviral genome and (b) inserting into the adenoviral genome a nucleic acid sequence DNA sequence comprising a promoter operatively linked to the virus transcription sequence; wherein the transcription sequence, when transcribed, produces a messenger RNA sequence that comprises a translatable sequence encoding a viral particle, and an untranslated sequence; wherein the untranslated sequence inhibits translation of the viral sequence under conditions that exist within normal mammalian cells that do not overexpress eukaryotic initiation factor eIF4E and wherein the untranslated sequence allows translation of the viral sequence under conditions that exist within mammalian cells that overexpress eukaryotic initiation factor eIF4E relative to normal cells.

As those of ordinary skill in the art will appreciate, the method provided by the present invention can include other steps or elements, such as the insertion of other nucleic acid sequences into, or deletion of such sequences from, the viral genome used to provide the viral vector. Furthermore, the various aspects of the present inventive method (e.g., the viral genome, nucleic acid sequences coding for 5′-UTR sequence, etc.) are as previously described herein with respect to the viral vector of the present invention.

The present inventive method of producing a viral vector can be carried out using techniques known to those of ordinary skill in the art. In general, virus vector construction relies on the high level of recombination between adenoviral nucleic acid sequences in a cell. Two or three separate viral nucleic acid sequences (e.g., DNA fragments), containing regions of similarity (or overlap) between sequences and constituting the entire length of the genome, are transfected into a cell. The host cell's recombination machinery constructs a full-length viral vector genome by recombining the aforementioned sequences. Other suitable procedures for constructing viruses containing alterations in various single regions have been previously described (Berkner et al., Nucleic Acids Res., 12, 925-941 (1984); Berkner et al., Nucleic Acids Res., 11, 6003-6020 (1983); Brough et al., Virol., 190, 624-634 (1992)) and can be used to construct multiply deficient viruses; yet other suitable procedures include, for example, in vitro recombination and ligation.

A preferred method of constructing the present inventive adenoviral vector first involves constructing the necessary deletions or modifications (such as adding a spacer element to a deleted region) of a particular region of the adenoviral genome. Such modifications can be performed, for example, in a plasmid cassette using standard molecular biological techniques. The altered nucleic acid sequence (containing the deletion or modification) then is moved into a much larger plasmid that contains up to one-half of the virus genome to provide a base plasmid comprising the modified adenoviral genome. The next step is to insert an expression cassette into a desired region of the modified adenoviral genome. The expression cassette can be provided by standard methods known in the art, for example, by isolating the cassette from a plasmid. The isolated cassette then can be transfected with the plasmid DNA (containing the modified adenoviral genome) into a recipient cell. The plasmid is, optionally, linearized prior to transfection by digestion with a suitable restriction enzyme to facilitate the insertion of the expression cassette at a desired position in the adenoviral genome. Selection of a suitable restriction enzyme is well within the skill of the ordinary artisan. The two pieces of DNA recombine to form a plasmid comprising the modified adenoviral genome and the expression cassette. The plasmid is isolated from the host cell and introduced into recipient cell that complements for the missing viral functions of the recombined viral genome to produce the adenoviral vector comprising the modified viral genome and the expression cassette. The vector can be further modified by alteration of the ITR and/or packaging signal.

Methods of Use

In one variation of the invention, the open reading frame for a virus capable of inducing apoptosis is functionally linked to regulatory DNA sequences in such a manner that the virus is constitutively expressed in a cell into which the recombinant virus is introduced. In this case, the expression of the virus is driven by a constitutive or stable promoter. The present invention does not dictate the choice of the stable promoter. The type of promoter is chosen to accomplish a useful expression profile in the context of the recombinant virus. Non-limiting examples of useful promoters for this variation of the invention include the Cytomegalovirus (CMV) immediate early promoter, The Simian Virus 40 (SV40) immediate early promoter, and promoters from eukaryotic household genes.

In another variation of the invention, the open reading frame is functionally linked to one or more control sequences, i.e. regulatory DNA sequences, in such a manner that the virus is only expressed or is expressed in a cell into which the recombinant adenovirus is introduced under certain conditions that can be modulated by an external signal, where the term “external” means having its origin outside of the DNA fragment encompassing the open reading frame and the regulatory DNA sequences. In this aspect of the invention, the expression of the virus is driven by a so-called regulatable or inducible promoter. Examples of the external signal include, but are not limited to, the addition or deprivation of a chemical compound, a shift in temperature, a decreased oxygen concentration, irradiation, and the like. Non-limiting examples of this kind of promoter include the heat shock protein 70 promoter, the promoter of an acute phase protein gene, such as the serum amyloid A3 gene or the complement factor 3 gene, the early growth response protein 1 promoter, the multidrug resistance gene 1 promoter, and promoters comprising one or more hypoxia-responsive elements, and fragments thereof (Kohno et al., Biochem. Biophys. Res. Comm. 165(1989):1415-1421; Varley et al., Proc. Natl. Acad. Sci. USA 92(1995):5346-5350; Hallahan et al., Nature Med. 1(1995):786-791; Dachs et al., Nature Med. 3(1997):515-520; Blackburn et al., Cancer Res. 58(1998):1358-1362; Binley et al., Gene Ther. 6(1999):1721-1727; Marples et al., Gene Ther. 7(2000):511-517). A special kind of regulatable promoter is a tissue- or cell type-specific promoter, where the external signal is provided by a protein that is only present in a particular type of cell or tissue. Non-limiting examples of tissue- or cell-type specific promoters are the prostate specific antigen promoter, the alpha-fetoprotein promoter, the albumin promoter, the carcinoembryonic antigen promoter, the cytokeratin 18 gene promoter, the kallikrein 2 promoter, the tyrosinase promoter, the osteocalcin promoter, the PAX-5 promoter and the alpha-lactalbumin promoter (Kaneko et al., Cancer Res. 55(1995):5283-5287; Richards et al., Hum. Gene Ther. 6(1995):881-893; Kozmik et al., Proc. Natl. Acad. Sci. USA 92(1995):5709-5713; Siders et al., Cancer Res. 56(1996):5638-5646; Chow et al., Proc. Natl. Acad. Sci. USA 94(1997):14695-14700; Shirakawa et al., Cancer Gene Ther. 5(1998):274-280; Gotoh et al., J. Urol. 160(1998):220-229; Anderson et al., Gene Ther. 6(1999):854-864; Yu et al., Cancer Res. 59(1999):4200-4203). Another special kind of regulatable promoter is a promoter that is responsive to an external signal that is provided by a protein that is not present in a particular type of cell or tissue. In particular, external signals that are absent in liver tissue are of interest in the context of in vivo administration of recombinant adenoviruses. Non-limiting examples of promoters that are responsive to external signals that are absent in liver tissue are the cyclooxygenase-2 promoter and the midkine promoter (Adachi et al., Cancer Res. 60(2000):4305-4310; Yamamoto et al., Mol. Ther. 3(2001):385-394). Another special kind of regulatable promoter is a promoter that is responsive to an external signal that is provided by a protein that is only present during a certain stage of the cell cycle. A non-limiting example of this kind of promoter is the promoter of a gene that is responsive to E2F, such as for example the adenovirus E2 gene or the E2F-1 gene. Another, not mutually excluding, special kind of regulatable promoter is a so-called transactivation response element (TRE). The TRE is a first component of a transactivation system that comprises as a second component a transactivator protein, that is capable of binding with specificity to the TRE, thereby regulating the transcription of a gene linked to the TRE.

In yet another variation of the invention, the open reading frame is functionally linked to regulatory DNA sequences in such a manner that the virus is only expressed in a cell into which the recombinant virus is introduced during the late phase of virus replication. Expression of the virus confined to the late phase of virus replication is of particular interest in the context of a CRAd. Since the replication will only occur in cells in which certain conditions exist that are exploited by the CRAd to allow the replication, expression of the virus will also be confined to the cells in which the certain conditions exist. This variation of the invention will thus add to the specificity of the CRAd. In this aspect of the invention, it is preferred that expression of the virus is driven by the adenovirus major late promoter (MLP). In recombinant adenoviruses according to the invention where the MLP drives expression of the open reading frame it is preferred that the expression cassette for the open reading frame comprises the in cis acting sequences required to confer full transcriptional activity of the MLP during the late phase of adenovirus replication as defined by Mondesart et al. (Nucleic Acids Res. 19(1991):3221-3228), included by reference herein. A useful expression cassette for this aspect of the invention was disclosed in U.S. Pat. No. 5,518,913, included by reference herein. Alternatively, the open reading frame is functionally linked to the endogenous MLP.

The present invention provides a method of treating a tumor or cancer in a host comprising administering an anti-cancer or anti-tumor effective amount of the viral vector of the present invention to a host in need thereof.

One skilled in the art will appreciate that suitable methods of administering a conditionally replicative viral vector of the present invention to an animal for therapeutic or prophylactic purposes (see, for example, Rosenfeld et al., Science, 252, 431-434 (1991), Jaffe et al., Clin. Res., 39(2), 302A (1991), Rosenfeld et al., Clin. Res., 39(2), 311A (1991), Berkner, BioTechniques, 6, 616-629 (1988)), are available, and, although more than one route can be used to administer the vector, a particular route can provide a more immediate and more effective reaction than another route.

The present invention provides a pharmaceutical composition comprising the viral vector of the present invention and a carrier, especially a pharmaceutically acceptable (e.g., a physiologically or pharmacologically acceptable) carrier (e.g., excipient or diluent). Pharmaceutically acceptable carriers are well-known to those who are skilled in the art and are readily available. The choice of carrier will be determined in part by the particular method used to administer the pharmaceutical composition. Accordingly, there is a wide variety of suitable formulations of the pharmaceutical composition of the present invention. The following formulations and methods are merely exemplary and are in no way limiting. However, oral, injectable and aerosol formulations are preferred.

Formulations suitable for oral administration can consist of (a) liquid solutions, such as an effective amount of the compound dissolved in diluents, such as water, saline, or orange juice; (b) capsules, sachets or tablets, each containing a predetermined amount of the active ingredient, as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The vectors of the present invention, alone or in combination with other suitable components, can be made into aerosol formulations to be administered via inhalation. These aerosol formulations can be placed into pressurized acceptable propellants, such as dichlorodifluoromethane, propane, nitrogen, and the like. They also can be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that refider the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Additionally, the vectors employed in the present invention can be made into suppositories by mixing with a variety of bases such as emulsifying bases or watersoluble bases. Formulations suitable for vaginal administration can be presented as pessaries, tampons, creams, gels, pastes, foams, or spray formulas containing, in addition to the active ingredient, such carriers as are known in the art to be appropriate.

The dose administered to an animal, particularly a human, in the context of the present invention will vary with the particular viral vector, the composition containing the viral vector, the method of administration, and the particular site and organism being treated. The dose should be sufficient to effect a desirable response, e.g., therapeutic or prophylactic response, within a desirable time frame.

The dose and dosage regimen will depend upon the nature of the cancer (primary or metastatic) and its population, the characteristics of the particular immunotoxin, e.g., its therapeutic index, the patient, the patient's history and other factors. The amount of virus administered will typically be in the range of about 10¹⁰ to about 10¹¹ viral particles per patient. The schedule will be continued to optimize effectiveness while balanced against negative effects of treatment. See Remington's Pharmaceutical Science, 17th Ed. (1990) Mark Publishing Co., Easton, Pa.; and Goodman and Gilman's: The Pharmacological Basis of Therapeutics 8th Ed (1990) Pergamon Press; which are incorporated herein by reference.

The present method of treating a tumor or cancer in a host further can comprise the administration (i.e., pre-administration, co-administration, and/or post-administration) of other treatments and/or agents to modify (e.g., enhance) the effectiveness thereof.

The replication deficient viral vectors of the present invention also have utility in vitro. For example, they can be used to study viral gene function and assembly, the production of TNF, or the expression of other foreign nucleic acid sequences in a suitable target cell. One of ordinary skill can identify a suitable target cell by selecting one that can be transfected by the viral vector, resulting in expression of the thereby inserted viral nucleic acid sequence complement. Preferably, a suitable target cell is selected that has receptors for attachment and penetration of virus into a cell. Such cells include, but are not limited to, those originally isolated from any mamma. Once the suitable target cell has been selected, the target cell is contacted with viral vector of the present invention, thereby effecting transfection or infection, respectively. Expression, toxicity, and other parameters relating to the insertion and activity of the nucleic acid sequence, or other foreign nucleic acid sequences, in the target cell then is measured using conventional methods well known in the art.

Moreover, cells explanted or removed from a patient having a disease that is suitably treated by gene therapy in the context of the present invention usefully are manipulated in vitro. For example, cells cultured in vitro from such an individual are placed in contact with viral vector of the present invention under suitable conditions to effect transfection, which are readily determined by one of ordinary skill in the art. Such contact suitably results in transfection of the vector into the cultured cells, where the transfected cells are selected using a suitable marker and selective culturing conditions. In so doing, using standard methods to test for vitality of the cells and thus measure toxicity and to test for presence of gene products of the foreign nucleic acid sequences of the vector of the present invention and thus measure expression, the cells of the individual are tested for compatibility with, expression in, and toxicity of the vector of the present invention, thereby providing information as to the appropriateness and efficacy of treatment of the individual with the vector system so tested. Such explanted and transfected cells, in addition to serving to test the potential efficacy/toxicity of a given gene therapy regime, also can be returned to an in vivo position within the body of the individual. Such cells so returned to the individual can be returned unaltered and unadorned except for the in vitro transfection thereof, or encased by or embedded in a matrix that keeps them separate from other tissues and cells of the individual's body. Such a matrix can be any suitable biocompatible material, including collagen, cellulose, and the like. Of course, alternatively or in addition, preferably after a positive response to the in vitro test, the transfection can be implemented in vivo by administration means as detailed hereinabove.

Further Aspects of the Present Invention

As those of ordinary skill in the art will appreciate, the viral vector, and methods involving the same, provided by the present invention can comprise any combination or permutation of the elements described herein.

However, many modifications and variations of the present illustrative nucleic acid sequence are possible. For example, the degeneracy of the genetic code allows for the substitution of nucleotides throughout coding regions, as well as in the translational stop signal, without alteration of the encoded polypeptide coding sequence. Such substitutable sequences can be deduced from the known amino acid or nucleic acid sequence of a given gene and can be constructed by conventional synthetic or site-specific mutagenesis procedures. Synthetic DNA methods can be carried out in substantial accordance with the procedures of Itakura et al., Science, 198, 1056-1063 (1977), and Crea et al., Proc. Natl. Acad. Sci. USA, 75, 5765-5769 (1978). Site-specific mutagenesis procedures are described in Maniatis et al., Molecular Cloning: A Laboratory Manual, Cold Spring Harbor, N.Y. (2d ed. 1989). Therefore, the present invention is in no way limited to the nucleic acid sequence specifically exemplified herein. Exemplified vectors are for therapy of tumors and/or cancer and, therefore, contain and express the viral gene. However, the vectors described also can comprise genes used to treat other similar or different diseases and/or afflictions including, but not limited to, other chronic lung diseases, such as emphysema, asthma, adult respiratory distress syndrome, and chronic bronchitis, as well as coronary heart disease, and other afflictions suitably treated or prevented by gene therapy, vaccination, and the like. Accordingly, any gene or nucleic acid sequence can be inserted into the viral vector.

The viral vector can be modified in other ways without departing from the scope and spirit of the present invention. For example, the coat protein of the present inventive viral vector can be manipulated to alter the binding specificity or recognition of the virus for a viral receptor on a potential host cell. Such manipulations can include deletion of regions of the fiber, penton, or hexon, insertions of various native or non-native ligands into portions of the coat protein, and the like. Manipulation of the coat protein can broaden the range of cells infected by a viral vector or enable targeting of a viral vector to a specific cell type.

For example, the vector can comprise a chimeric coat protein (e.g., a fiber, hexon or penton protein), which differs from the wild-type (i.e., native) coat protein by the introduction of a nonnative amino acid sequence, preferably at or near the carboxyl terminus. Preferably, the nonnative amino acid sequence is inserted into or in place of an internal coat protein sequence. The resultant chimeric viral coat protein is able to direct entry into cells of the viral vector comprising the coat protein that is more efficient than entry into cells of viral vector that is identical except for comprising a wild-type viral coat protein rather than the chimeric viral coat protein.

The chimeric virus coat protein desirably binds a novel endogenous binding site present on the cell surface. A result of this increased efficiency of entry is that the viral virus can bind to and enter numerous cell types which a virus comprising wild-type coat protein typically cannot enter or can enter with only a low efficiency.

Alternatively, the viral vector of the present invention can comprise a chimeric virus coat protein that is not selective for a specific type of eukaryotic cell. Such chimeric coat protein differs from the wild-type coat protein by an insertion of a nonnative amino acid sequence into or in place of an internal coat protein sequence. In a vector comprising a non-selective chimeric coat protein, the virus coat efficiently binds to a broader range of eukaryotic cells than a wild-type virus coat, such as described in International Patent Application WO 97/20051.

Specificity of binding of virus to a given cell also can be adjusted by use of virus comprising a short-shafted viral fiber gene, as discussed in U.S. Pat. No. 5,962,311. Use of virus comprising a short-shafted viral fiber gene reduces the level or efficiency of viral fiber binding to its cell-surface receptor and increases viral penton base binding to its cell-surface receptor, thereby increasing the specificity of binding of the virus to a given cell. Alternatively, use of virus comprising a short-shafted fiber enables targeting of the virus to a desired cell-surface receptor by the introduction of a nonnative amino acid sequence either into the penton base or the fiber knob.

In addition, the ability of a viral vector to recognize a potential host cell can be modulated without genetic manipulation of the coat protein. For instance, complexing virus with a bispecific molecule comprising a penton base-binding domain and a domain that selectively binds a particular cell surface binding site enables one of ordinary skill in the art to target the vector to a particular cell type.

Many modifications to a viral vector, specifically viral vector, are known in the art. Suitable modifications for viral vector include those modifications described in U.S. Pat. Nos. 5,559,099; 5,731,190; 5,712,136; 5,770,442; 5,846,782; 5,926,311; and 5,965,541 and International Patent Applications WO 96/07734, WO 96/26281, WO 97/20051, WO 98/07865, WO 98/07877, and WO 98/54346.

The virus may be modified by incorporation of mutated coat proteins into the virion outer capsid. In one embodiment, the virus is a virus modified to reduce or eliminate an immune reaction to the virus. Such modified virus are termed “immunoprotected virus”. Such modifications could include packaging of the virus in a liposome, a micelle or other vehicle to mask the virus from the mammals immune system.

Neoplasms that are particularly susceptible to treatment by the methods of the invention include breast cancer, central nervous system cancer (e.g., neuroblastoma and glioblastoma), peripheral nervous system cancer, lung cancer, prostate cancer, colorectal cancer, thyroid cancer, renal cancer, adrenal cancer, liver cancer, lymphoma and leukemia..

The virus is administered to a proliferating cell or neoplasm in a manner so that it contacts the proliferating cells or cells of the neoplasm or neoplastic cells. The route by which the virus is administered, as well as the formulation, carrier or vehicle, will depend on the location as well as the type of the neoplasm. A wide variety of administration routes can be employed. For example, for a solid neoplasm that is accessible, the virus can be administered by injection directly to the neoplasm or a hematopoietic neoplasm, for example, the virus can be administered intravenously or intravascularly. For neoplasms that are not easily accessible within the body, such as metastases or brain tumors, the virus is administered in a manner such that it can be transported systemically through the body of the mammal and thereby reach the neoplasm (e.g., intrathecally, intravenously or intramuscularly).

Alternatively, the virus can be administered directly to a single solid neoplasm, where it then is carried systemically through the body to metastases. The virus can also be administered subcutaneously, intraperitoneally, topically (e.g., for melanoma), orally (e.g., for oral or esophageal neoplasm), rectally (e.g., for colorectal neoplasm), vaginally (e.g., for cervical or vaginal neoplasm), nasally or by inhalation spray (e.g., for lung neoplasm).

Virus can be administered systemically to mammals which are immune compromised or which have not developed immunity to the virus epitopes. In such cases, virus administered systemically, i.e. by intraveneous injection, will contact the proliferating cells resulting in lysis of the cells.

Immunocompetent mammals previously exposed to a particular virus, such as modified adenovirus, modified HSV, modified vaccinia virus and modified parapoxvirus orf virus, may have developed humoral and/or cellular immunity to that virus. Nevertheless, it is contemplated that direct injection of the virus into a solid tumor in immunocompetent mammals will result in the lysis of the neoplastic cells.

When the virus is administered systemically to immunocompetent mammals, the mammals may produce an immune response to the virus. Such an immune response may be avoided if the virus is of a subtype to which the mammal has not developed immunity, or the virus has been modified as previously described herein such that it is immunoprotected, for example, by protease digestion of the outer capsid or packaging in a micelle.

It is contemplated that the virus may be administered to immunocompetent mammals immunized against the virus in conjunction with the administration of anti-antivirus antibodies. Such anti-antivirus antibodies may be administered prior to, at the same time or shortly after the administration of the virus. Preferably an effective amount of the anti-antivirus antibodies are administered in sufficient time to reduce or eliminate an immune response by the mammal to the administered virus.

Alternatively, it is contemplated that the immunocompetency of the mammal against the virus may be suppressed either by the prior or co-administration of pharmaceuticals known in the art to suppress the immune system in general or alternatively the administration of such immunoinhibitors as anti-antivirus antibodies.

The humoral immunity of the mammal against the virus may also be temporarily reduced or suppressed by plasmaphoresis of the mammals blood to remove the anti-virus antibodies. The anti-virus antibodies removed by this process correspond to the virus selected for administration to the patient.

Other agents are known to have immunosuppressant properties as well (see, e.g., Goodman and Gilman, 7th Edition, page 1242, the disclosure of which is incorporated herein by reference. Such immunoinhibitors also include anti-antivirus antibodies, which are antibodies directed against anti-virus antibodies.

Such anti-antivirus antibodies may be administered prior to, at the same time or shortly after the administration of the virus. Preferably an effective amount of the anti-antivirus antibodies are administered in sufficient time to reduce or eliminate an immune response by the mammal to the administered virus.

In yet other methods of the invention, a virus selected from the group consisting of modified adenovirus, modified HSV, modified vaccinia virus and modified parapoxvirus orf virus is administered to proliferating cells in the individual mammal. In one embodiment of this invention a course of this therapy is administered one or more times. Following the first administration of virus therapy particular immune constituents that may interfere with subsequent administrations of virus are removed from the patient. These immune constituents include B cells, T cells, antibodies, and the like.

Removal of either the B cell or T cell population can be accomplished by several methods. In one method, the blood may be filtered and heme-dialysis may be performed. Another method is the filtration of the blood coupled with extra corporeal compounds that can remove the cell populations, for example, with immobilized antibodies that recognize specific receptors on the cell population which is to be remove. Yet another method for removal of a cell population is by immune suppression. This can be done by first line radiation therapy or by cyclic steroids such as cyclosporin. Selective removal of anti-virus antibodies can also prevent the patient's immune system from removing therapeutically administered virus. Preventing antibody interaction with the administered virus may also assist systemic treatment strategies.

The following examples further illustrate the present invention and, of course, should not be construed as in any way limiting its scope. Many of the techniques employed herein are well known to those in the art. Molecular biology techniques are described in detail in suitable laboratory manuals, such as Maniatis et al., Molecular Cloning: A Laboratory Manual, Cold Spring Harbor, N.Y. (2d ed. 1989), and Current Protocols in Molecular Biology (Ausubel et al., eds. (1987)).

### LITERATURE CITED

1. De Benedetti A, Graff JR. eIF4E expression and its role in malignancies and metastases. Oncogene. 23: 3189-3199. Review, (2004).
2. DeFatta R J, Chervenak R P, De Benedetti A. A cancer gene therapy approach through translational control of a suicide gene. Cancer Gene Ther. 9:505-512, (2002).
3. DeFatta R J, Li Y, De Benedetti A. Selective killing of cancer cells based on translational control of a suicide gene. (2002), Cancer Gene Ther. 9:573-578.
4. Reid, T. et al. Hepatic arterial infusion of a replication-selective oncolytic adenovirus (dl 1520): phase II viral, immunologic, and clinical endpoints. Cancer Res. 62: 6070-6079, (2002).
5. Asada, T. Treatment of human cancer with mumps virus. Cancer, 34:1907-28 (1974).
6. Okuno, Y. et al. Studies on the use of mumps virus for treatment of human cancer. Biken J. 21:37-49, (1978).
7. Webb, H. E. & Smith, C. E. Viruses in the treatment of cancer. Lancet 1: 1206-1208 (1970).
8. Dupressoir, T., Vanacker, J. M., Cornelis, J. J., Duponchel, N. & Rommelaere, J. Inhibition by parvovirus H-1 of the formation of tumors in nude mice and colonies in vitro by transformed human mammary epithelial cells. Cancer Res. 49:3203-2308 (1989).
9. Faisst, S. et al. Dose-dependent regression of HeLa cell-derived tumours in SCID mice after parvovirus H-1 infection. Int. J. Cancer 75: 584-589, (1998).
10. Mineta, T., Rabkin, S. D. & Martuza, R. L. Treatment of malignant gliomas using ganciclovir-hypersensitive, ribonucleotide reductase-deficient herpes simplex viral mutant. Cancer Res. 54:3963-6 (1994).
11. Mineta, T., Rabkin, S. D., Yazaki, T., Hunter, W. D. & Martuza, R. L. Attenuated multi-mutated herpes simplex virus-i for the treatment of malignant gliomas. Nat. Med. 1:938-43, (1995).
12. Bischoff, J. R., Kim, D. H., Williams, A., Heise, C., et al. An adenovirus mutant, that replicates selectively in p53-deficient human tumor cells. Science, 274:373-376, (1996).
13. Heise, C., Ganly, I., Kim, Y. T., Sampson-Johannes, A., Brown, R., Kim, D. et al. Efficacy of a replication-selective adenovirus against ovarian carcinomatosis is dependent on tumor burden, viral replication and p53 status. Gene Ther. 7: 1925-1929 (2000).
14. Heise, C. C., Williams, A. M., Xue, S., Propst, M. & Kim, D. H. Intravenous administration of ONYX-015, a selectively replicating adenovirus, induces antitumoral efficacy. Cancer Res. 59: 2623-2628, (1999).
15. Ganly, I., Kim, D., Eckhardt, G., Rodriguez, G. I., et al. A phase I study of Onyx-015, an E1B attenuated adenovirus, administered intratumorally to patients with recurrent head and neck cancer. Clin. Cancer Res. 6: 798-806, (2000).
16. Babiss, L. E., Friedman, J. M. & Damell, J. E., Jr. Cellular promoters incorporated into the adenovirus genome: effects of viral regulatory elements on transcription rates and cell specificity of albumin and beta-globin promoters. Mol. Cell Biol. 6, 3798-806, (1986).
17. Muller, A. Homey, B., Soto, H., Ge, N., et al. Involvement of chemokine receptors in breast cancer metastasis. Nature, 410: 50-56 (2001).
18. Taichman, R. S., Cooper, C., Keller, E. T., Pienta, K. J., Taichman, N. S., McCauley, L. K., Use of the stromal cell-derived factor-1/CXCR4 pathway in prostate cancer metastasis to bone. Cancer Res. 62:1832-1837, (2002).
19. Sanz-Rodriguez, F., Hidalgo, A. & Teixido, J. Chemokine stromal cell-derived factor-1 alpha modulates VLA-4 integrin-mediated multiple myeloma cell adhesion to CS-1/fibronectin and VCAM-1, Blood 97: 346-351 (2001).
20. Arai, J., Yasukawa, M., Yakushijin, Y., Miyazaki, T. & Fujita, S. Stromal cells in lymph nodes attract B-lymphoma cells via production of stromal cell-derived factor-1. Eur. J Haematol. 64,323-332, (2000).
21. Zhu, Z. B., Makhija, S. K., Lu, B., et al. Transcriptional targeting of adenoviral vector through the CXCR4 tumor-specific promoter. Gene Ther, 11, 645-648, (2004).
22. Mathews M Translational Control. CSH Press. (1989).
23. Trachsel H Translation in Eukaryotes. CRC Press. (1991).
24. Hershey J W B, Mathews M B, and Sonenberg N Translational Control. CSH Press. (1996).
25. Hiremath, L. S., N. R. Webb, and R. E. Rhoads Immunological detection of the messenger RNA cap-binding protein. J. Biol. Chem. 260:7843-7849, (1985)
26. Duncan. R., S. C. Milburn, and J. B. Hershey Regulated phosphorylation and low abundance of HeLa cell initiation factor eIF-4 suggests a role in translational control. J. Biol. Chem. 262:380-388. (1987)
27. De Benedetti, A., Joshi-Barve, S., Rinker-Schaeffer, C., and Rhoads, R. E. Expression of antisense RNA against initiation factor eIF4E mRNA in HeLa cells results in lengthened cell division times, diminished translation rates, and reduced levels of both eIF4E and the p220 component of eIF-4F. Mol. Cell. Biol.11: 5435-5445. (1991).
28. Lazaris-Karatzas, A., K. S. Montine, & N. Soneneberg, Malignant transformation by a eukaryotic initiation factor subunit that binds to mRNA 5′ cap. Nature, 345: 544-547. (1990).
29. De Benedetti, A. and Rhoads, R. E. Overexpression of eukaryotic protein synthesis initiation factor 4E in HeLa cells results in aberrant growth and morphology. Proc. Natl. Acad. Sci. USA, 87: 8212-8216.
30. De Benedetti, A. and Harris, A. L. (1999) eIF4E expression in tumors: its possible role in progression of malignancies. Int.J.Biochem.Mol.Biol.31: 59-72. (1990).
31. Nathan C A, Carter P, Liu L, Li B D, Abreo F, Tudor A, Zimmer S G, and De Benedetti A Elevated expression of eIF4E and FGF2 isoforms during vascularization of breast carcinomas. Oncogene. 15:1087-1094, (1997).
32. Lodish H F Model for the regulation of mRNA translation applied to haemoglobin synthesis. Nature (London), 251: 385-388, (1974).
33. Baserga R The biology of cell reproduction. Harvard Un. Press. (1985).
34. Baserga R The cell cycle: myths and realities. Cancer Research. 50:6769-6771. (1990).
35. Rhoads, R. E. Cap recognition and the entry of mRNA into the protein synthesis initiation cycle. TIBS. 13: 52-56, (1988).
36. Thach, R. E. Cap recap: the involvement of eIF4E in regulating gene expression. Cell 22:177-180. (1992).
37. Rhoads, R. E. Protein synthesis, cell growth and oncogenesis. Curr. Opin. Cell Biol. 3: 1019-1024. (1991).
38. Rhoads, R. E. Regulation of eukaryotic protein synthesis by initiation factors. J. Biol. Chem. 266:3017-3020, (1993).
39. Morley, S. J. Signal transduction mechanisms in the regulation of protein synthesis. Mol. Biol. Reports 19: 221-231. (1994).
40. Sonenberg, N. Remarks on the mechanism of ribosome binding to eukaryotic mRNAs. Gene Expression 3: 317-32, (1993).
41. Pelletier, J., and N. Sonenberg. The involvement of mRNA secondary structure in protein synthesis. Biochem. Cell Biol. 65:576-581, (1987).
42. Kozak, M., An analysis of 5′-noncoding sequences from 699 vertebrate messenger RNAs. Nuceic. Acid. Res.15: 8125-8147. (1989).
43. Geballe, A. P., and Morris, D. R. Initiation codons within 5′-leaders of mRNAs as regulators of translation. TIBS. 19:150-164, (1994).
44. Kozak, M. An analysis of vertebrate mRNA sequences: intimations of translational control. J. Cell Biol. 115:887-903. (1991).
45. Ruan, H., L M. Shantz, A E. Pegg, and R. Morris The upstream open reading frame of the mRNA encoding S-Adenosylmethionine decarboxylase is a polyamine-responsive translational control element. J.Biol.Chem.271: 29576-29582, (1996).
46. Folkman J., What is the evidence that tumors are angiogenesis dependent? J. Natl. Cancer Inst. 84: 4-6, (1990).
47. Folkman J. and Klagsbrun M., Angiogenic factors. Science. 235:442-447, (1987).
48. Kevil C., Carter, P., Hu, B., and De Benedetti, A. Translational Enhancement of FGF2 by eIF-4 factors, and alternate utilization of CUG and AUG codons for translation initiation. Oncogene. 11:2339-2348, (1995).
49. Kevil, C., De Benedetti, A., Payne, K. D., Coe, L. L., Laroux, S., and Alexander, S., Translational regulation of Vascular Permeability Factor by eukaryotic initiation factor 4E: Implications for tumor angiogenesis. International J. Cancer. 65:785-790, (1996).
50. Scott, PA E, Smith, K., Poulsom, R., De Benedetti, A., Bicknell, R., Harris, A L., Differential expression of vascular endothelial growth factor mRNA versus protein isoforms expression in human breast cancer and relationship to eIF4E. British. J. Cancer.77: 2120-2128, (1998).
51. Goto F, Goto K, Weindel K., and Folkman J Synergistic effects of vascular endothelial growth factor and basic fibroblast growth factor on the proliferation and cord formation of bovine capillary endothelial cells within collagen gels. Lab Investigation. 69:508-517, (1993).
52. DeFatta R J, Turbat-Herrera E A, Li B D L, Anderson W, and De Benedetti A Elevated expression of eIF4E in confined early breast cancer lesions: possible role of hypoxia. Int. J. Cancer. 80:516-522, (1999).
53. Rosenwald, I B, Chen, J J, Wang, S, Svas, L, London, I M, Pullman, J Upregulation of protein synthesis initiation factor eIF4E is an early event in colon carcinogenesis. Oncogene, 18: 2507-2517, (1999).
54. Miyagi, Y., A. Sugiyama, A. Asai, T. Okzaki, Y. Kuchino, S. Kerr Elevated levels of eukaryotic initiation factor eIF4E mRNA in a broad spectrum of transformed cell lines. Cancer Letters 91: 247-252, (1995).
55. Anthony, B., Carter, P., and De Benedetti, A. Overexpression of the proto-oncogene-translation factor eIF4E in breast carcinoma cell lines. Int. J. Cancer. 65:858-863, (1996).
56. Bauerschmitz G J, Kanerva A, Wang M, Herrmann I, Shaw D R, Strong T V, Desmond R, Rein D T, Dall P, Curiel D T, Hemminki A. Evaluation of a selectively oncolytic adenovirus for local and systemic treatment of cervical cancer. Int. J. Cancer. 111:303-309, (2004).
57. Redenbacher M, Rein D T, Wang M, Nettelbeck D M, Hemminki A, Ulasov I, Rivera A R, Everts M, Alvarez R D, Douglas J T, Curiel D T. Genetic replacement of the adenovirus shaft fiber reduces liver tropism in ovarian cancer gene therapy. Hum Gene Ther. 15:509-18. (2004).
58. Volk A L, Rivera A A, Kanerva A, Bauerschmitz G, Dmitriev I, Nettelbeck D M, Curiel D T. Enhanced adenovirus infection of melanoma cells by fiber-modification: incorporation of RGD peptide or Ad5/3 chimerism. Cancer Biol Ther. 2:511-5. (2003).
59. Hemminki A, Wang M, Desmond R A, Strong T V, Alvarez R D, Curiel D T. Serum and ascites neutralizing antibodies in ovarian cancer patients treated with intraperitoneal adenoviral gene therapy. Hum. Gene Ther. 13:1505-14. (2002).
60. Alvarez R D, Gomez-Navarro J, Wang M, Barnes M N, Strong T V, Arani R B, Arafat W, Hughes J V, Siegal G P, Curiel D T. Adenoviral-mediated suicide gene therapy for ovarian cancer. Mol. Ther. 2:524-30. (2000).
61. Alvarez R D, Barnes M N, Gomez-Navarro J, Wang M, Strong T V, Arafat W, Arani R B, Johnson M R, Roberts B L, Siegal G P, Curiel D T. A cancer gene therapy approach utilizing an anti-erbB-2 single-chain antibody-encoding adenovirus (AD21): a phase 1 trial. Clin. Cancer Res. 6:3081-3087, (2000).
62. Coppola, D R New Biolnnovation center jazzes up New Orleans' technology—In the field: pharmaceutical science & technology news—New Orleans Biolnnovation Center Pharmaceutical Technology, (2004).
63. Crowe D L, Hacia J G, Hsieh C L, Sinha U K, Rice H. Molecular pathology of head and neck cancer. Histol Histopathol. 17:909-914, (2002).
64. Kim E S, Glisson B S. Treatment of metastatic head and neck cancer: chemotherapy and novel agents. Cancer Treat. Res. 114:295-314, (2003).
65. Pearson S, Jia H, Kandachi K. China approves first gene therapy. Nat. Biotechnol. 22:3-4, (2004).
66. Anonymous. INGN 201: Ad-p53, Ad5CMV-p53, Adenoviral p53, INGN 101, p53 gene therapy—Introgen, RPR/INGN 201. BioDrugs. 17:216-222, (2003).

1. A conditionally replicating recombinant virus vector, comprising a replicating recombinant virus vector genome, comprising: (a) a replicating recombinant virus vector genome nucleic acid transcription sequence; and (b) a control sequence operatively linked to the transcription sequence; wherein the transcription sequence, when transcribed, produces a messenger RNA sequence that comprises an open reading frame encoding at least one viral protein necessary for replication, and a 5′-untranslated region (5′-UTR) sequence; wherein the untranslated sequence inhibits translation of the viral protein sequence under conditions that exist within normal mammalian cells that do not overexpress eukaryotic initiation factor eIF4E; and wherein the untranslated sequence allows translation of the viral protein sequence under conditions that exist within mammalian cells that overexpress eukaryotic initiation factor eIF4E relative to normal cells.

2. The composition of claim 1 wherein the untranslated sequence further comprises a secondary structure conformation having a stability measured as folded state free energy of ΔG≦ about −50 Kcal/Mol.

3. The composition of claim 1 wherein control sequence is a promoter.

4. The composition of claim 3 wherein the promoter is a tissue-specific promoter.

5. The composition of claim 3 wherein the promoter is an inducible promoter.

6. The composition of claim 1 wherein the virus vector genome comprises an adenovirus.

7. The composition of claim 1 wherein the virus vector genome comprises a type 2 or type 5 virus vector.

8. The composition of claim 1 wherein the virus vector genome is a type 2 or type 5 virus vector (Ad5) comprising an E1A gene operatively linked to the 5′-UTR sequence.

9. The composition of claim 2 wherein the 5′-UTR sequence is an oligonucteotide comprising at least a self-complementary sequence.

10. The composition of claim 9 wherein the 5′-UTR sequence comprises at least 30 nucleotides.

11. The composition of claim 10 wherein the 5′-UTR sequence is derived from sequences on genes selected from the group consisting of Fibroblast Growth Factor 2 (FGF2), proto-oncogene c-myc, cyclinD1, ornithine decarboxylase, and vascular endothelial growth factor (“VEGF”) .

12. The composition of claim 10 wherein the 5′-UTR sequence is derived from a Fibroblast Growth Factor 2 (FGF2) coding sequence.

13. The composition of claim 8 wherein the 5′-UTR sequence is upstream of the E1A mRNA coding sequence.

14. The composition of claim 1 wherein the viral genome codes for an oncolytic virus.

15. The composition of claim 1 wherein the virus is selected from the group consisting of adenovirus, HSV, vaccinia virus and parapoxvirus orf virus.

16. The composition of claim 1 wherein the virus is a recombinant virus from two or more types of viruses with differing pathogenic phenotypes such that it contains different antigenic determinants.

17. The composition of claim 1 wherein the viral genome is a recombinant adenovirus vector genome.

18. The composition of claim 17 wherein the recombinant adenovirus vector genome is encapsidated within an adenovirus capsid.

19. The composition of claim 18 wherein the adenovirus vector genome comprises AAV cap sequences.

20. The composition of claim 19 wherein the vector genome comprises from about 2.8 kb to 38 kb.

21. The composition of claim 17 wherein the virus vector genome comprises an early gene coding region operatively associated with a promoter selected from the group consisting of liver-specific, skeletal muscle-specific, cardiac muscle-specific, smooth muscle-specific, diaphragm muscle-specific, prostate-specific, and brain-specific promoters.

21. The composition of claim 17 wherein the virus vector genome comprises an early gene coding region operatively associated with a cancer cell specific promoter.

22. The composition of claim 1 wherein the virus vector genome comprises an adenovirus early gene coding region selected from the group consisting of E1, E2, E3, and E4 operatively associated with a cancer cell specific promoter.

23. The composition of claim 1 wherein the control sequence is a tissue-specific promoter derived from genes encoding at least one protein selected from the group consting of the prostate specific antigen (PSA), Carcinoembryonic antigen (CEA), secretory leukoprotease inhibitor (SLPI), alpha-fetoprotein (AFP), vascular endothelial growth factor, CXCR4 and survivin.

24. The composition of claim 23 wherein the tissue-specific promoter is an inducible promoter.

25. The composition of claim 23 wherein the tissue-specific promoter is the CXCR4 promoter.

26. The composition of claim 1 wherein the virus vector genome comprises an E1 coding region operatively associated with an inducible promoter.

27. The composition of claim 1 wherein the vector, when administered to a cell that overexpresses eIF4E, is effective to inhibit cell growth or lyse the cell.

28. The composition of claim 1 wherein the untranslated sequence allows translation of the viral sequence under conditions that exist within mammalian cells that overexpress eukaryotic initiation factor eIF4E at least 2-fold greater relative to normal cells.

29. The composition of claim 28 wherein the cell is a metastatic tumor cell.

30. The composition of claim 28 wherein the cell is a solid tumor cell.

31. The composition of claim 29 wherein the metastatic tumor cellis associated with a mammalian cancer selected from the group consisting of bladder, breast, cervical, colon, lung, prostate, and head and neck.

32. The composition of claim 1 wherein the virus vector genome further comprises a pharmaceutically acceptable carrier.

32. The composition of claim 1 wherein the virus vector genome further comprises a pharmaceutically acceptable carrier.

33. A cultured cell comprising the conditionally replicating recombinant virus vector of claim 1.

34. A pharmaceutical composition comprising the conditionally replicating recombinant virus vector of claim 1 and a pharmaceutically acceptable excipient.

35. A pharmaceutical composition comprising the conditionally replicating recombinant virus vector of claim 1, a chemotherapeutic agent and a pharmaceutically acceptable excipient.

36. A method for conditionally expressing a replicating recombinant virus vector in a cell, comprising administering to the cell a DNA sequence comprising a replicating recombinant virus vector genome, comprising: (a) a replicating recombinant virus vector genome nucleic acid transcription sequence; and (b) a control sequence operatively linked to the transcription sequence; wherein the transcription sequence, when transcribed, produces a messenger RNA sequence that comprises an open reading frame encoding at least one viral protein necessary for replication, and a 5′-untranslated region (5′-UTR) sequence; wherein the untranslated sequence inhibits translation of the viral protein sequence under conditions that exist within normal mammalian cells that do not overexpress eukaryotic initiation factor eIF4E; and wherein the untranslated sequence allows translation of the viral protein sequence under conditions that exist within mammalian cells that overexpress eukaryotic initiation factor eIF4E relative to normal cells.

37. The method of claim 36, wherein the untranslated sequence further comprises a hairpin secondary structure conformation having a stability measured as folded state free energy of ΔG≦ about −50 Kcal/Mol.

38. The method of claim 36, wherein the untranslated sequence allows translation of the toxin sequence under conditions that exist within mammalian cells that overexpress eukaryotic initiation factor eIF4E at least 2-fold greater relative to normal cells.

39. The method of claim 36 wherein the administering is in an amount effective to inhibit cell growth.

40. The method of claim 36, wherein the expression vector is delivered within a liposomal construct.

41. A method for conditionally expressing a replicating recombinant virus vector in a cell, comprising administering to the cell an RNA sequence comprising a translatable messenger RNA sequence that comprises an open reading frame encoding at least one viral protein necessary for replication, and a 5′-untranslated region (5′-UTR) sequence; wherein the untranslated sequence inhibits translation of the viral protein sequence under conditions that exist within normal mammalian cells that do not overexpress eukaryotic initiation factor eIF4E; and wherein the untranslated sequence allows translation of the viral protein sequence under conditions that exist within mammalian cells that overexpress eukaryotic initiation factor eIF4E relative to normal cells.

42. The method of claim 41, wherein the untranslated sequence further comprises a hairpin secondary structure conformation having a stability measured as folded state free energy of ΔG≦ about −50 Kcal/Mol.

43. A method of treatment for a proliferation disease in a mammal, comprising administering to a mammal in need of such treatment a therapeutically effective amount of a DNA sequence comprising a replicating recombinant virus vector genome, comprising: (a) a replicating recombinant virus vector genome nucleic acid transcription sequence; and (b) a control sequence operatively linked to the transcription sequence; wherein the transcription sequence, when transcribed, produces a messenger RNA sequence that comprises an open reading frame encoding at least one viral protein necessary for replication, and a 5′-untranslated region (5′-UTR) sequence; wherein the untranslated sequence inhibits translation of the viral protein sequence under conditions that exist within normal mammalian cells that do not overexpress eukaryotic initiation factor eIF4E; and wherein the untranslated sequence allows translation of the viral protein sequence under conditions that exist within mammalian cells that overexpress eukaryotic initiation factor eIF4E relative to normal cells.

44. The method as recited in claim 43, wherein the untranslated sequence further comprises a hairpin secondary structure conformation having a stability measured as folded state free energy of ΔG≦ about -50 Kcal/Mol.

45. The method of claim 44, wherein the untranslated sequence allows translation of the sequence under conditions that exist within tumor cells that overexpress eukaryotic initiation factor eIF4E at least 2-fold greater relative to normal cells.

46. The method of claim 44, wherein the untranslated sequence allows translation of the vial sequence within tumor cells in which the presence of eukaryotic initiation factor eIF4E allows the translation of the sequence, the viral genome is translated to produce viruses capable of substantial lyses the tumor cells.

47. The method of claim 44, wherein the majority of non-tumor cells in the mammal are not killed due to the low levels of eukaryotic initiation factor eIF4E typically present in non-tumor cells

48. The method of claim 44, wherein the encoded virus is a conditional virus.

49. The method of claim 44, wherein the method additionally comprises administering an effective amount of a chemotherapeutic agent to the mammal.

50. The method of claim 44, wherein the cancer is a metastatic tumor.

51. The method of claim 44, wherein the cancer is a solid tumor.

52. The method of claim 50, wherein the metastatic tumor is associated with a mammalian cancer selected from the group consisting of bladder, breast, cervical, colon, lung, prostate, and head and neck.

53. A method of treatment for cancer in a mammal, comprising administering to a mammal in need of such treatment a therapeutically effective amount of a messenger RNA sequence that comprises a translatable sequence comprising an open reading frame encoding at least one viral protein necessary for replication, and a 5′-untranslated region (5′-UTR) sequence; wherein the untranslated sequence inhibits translation of the viral protein sequence under conditions that exist within normal mammalian cells that do not overexpress eukaryotic initiation factor eIF4E; and wherein the untranslated sequence allows translation of the viral protein sequence under conditions that exist within mammalian cells that overexpress eukaryotic initiation factor eIF4E relative to normal cells

54. The method as recited in claim 53, wherein the untranslated sequence further comprises a hairpin secondary structure conformation having a stability measured as folded state free energy of ΔG≦ about −50 Kcal/Mol.

55. The method of claim 54 wherein control sequence is a promoter.

56. The method of claim 55 wherein the promoter is a tissue-specific promoter.

57. The method of claim 55 wherein the promoter is an inducible promoter.

58. The method of claim 55 wherein the virus vector genome comprises an adenovirus.

59. The method of claim 55 wherein the virus vector genome comprises a type 2 or type 5 virus vector.

60. The method of claim 55 wherein the virus vector genome is a type 2 or type 5 virus vector (Ad5) comprising an E1A gene operatively linked to the 5′-UTR sequence.

61. The method of claim 55 wherein the 5′-UTR sequence is upstream of the E1A mRNA coding sequence.

62. The method of claim 55 wherein the viral genome codes for an oncolytic virus.

63. The method of claim 55 wherein the virus is a recombinant virus from two or more types of viruses with differing pathogenic phenotypes such that it contains different antigenic determinants.

64. The method of claim 55 wherein the viral genome is a recombinant adenovirus vector genome.

65. The method of claim 55 wherein the recombinant adenovirus vector genome is encapsidated within an adenovirus capsid.

66. The method of claim 55 wherein the virus vector genome comprises an early gene coding region operatively associated with a promoter selected from the group consisting of liver-specific, skeletal muscle-specific, cardiac muscle-specific, smooth muscle-specific, diaphragm muscle-specific, prostate-specific, and brain-specific promoters.

67. The method of claim 55 wherein the virus vector genome comprises an early gene coding region operatively associated with a cancer cell specific promoter.

68. The method of claim 55 wherein the virus vector genome comprises an adenovirus early gene coding region selected from the group consisting of E1, E2, E3, and E4 operatively associated with a cancer cell specific promoter.

69. The method of claim 55 wherein the control sequence is a tissue-specific promoter derived from genes encoding at least one protein selected from the group consting of the prostate specific antigen (PSA), Carcinoembryonic antigen (CEA), secretory leukoprotease inhibitor (SLPI), alpha-fetoprotein (AFP), vascular endothelial growth factor, CXCR4 and survivin.

70. The method of claim 23 wherein the tissue-specific promoter is the CXCR4 promoter.
